SEQUENCE LISTING (60 sequences)

SEQ ID NO: 1 - DNA, 1170 bp, Bacillus licheniformis
  CDS alr
atgagcttaaaaccattctatagaaagacatgggccgaaatcgatttaacggctttaaaagaaaacgtccgcaatatgaagcggcacatcggcgagcatgtccgcctgatggccgtcgttaaagcgaatgcctacggacacggggatgcacaggtagcgaaggcggctcttgcagaaggggcgtccattcttgctgtggctttattggatgaagcgctttcgctgagggcgcaggggattgaagaaccgattcttgtcctcggtgcagtgccgaccgaatatgcaagcattgccgcggaaaagcgcattatcgtgactggctactccgtcggctggctgaaagacgtgctcggttttctgaatgaggccgaagctcctcttgaatatcatttgaagatcgacacgggcatgggccgccttggctgcaaaacggaagaagagatcaaagaaatgatggagatgaccgaatcgaacgataagctcaattgtacgggcgtgttcactcatttcgccacggcggacgaaaaggacaccgattatttcaacatgcagcttgaccgctttaaagagctgatcagccccctcccgcttgaccgtttgatggtgcattcgtcaaacagcgccgcgggtctgcgcttcagggaacagctatttaatgccgtccgcttcggcatcggcatgtacggtttggcgccgtcaaccgaaataaaagacgagctgccgtttcgtctgcgggaagtgttttcgcttcataccgaactcacccatgtgaaaaaaattaaaaaaggcgagagcgtcagctacggggcgacatatacagctcagcgcgacgaatggatcgggacagtccccgtcgggtatgccgacggatggctgaggcgcctggccggaacggaagtgctgatcgacggaaaacgccaaaaaatagcagggagaatctgcatggaccagttcatgatttcccttgccgaagaataccctgtcggcacaaaggttaccttgatcggaaagcaaaaagacgaatggatctcagtcgacgaaatcgcccaaaatttgcagacgatcaattatgaaattacctgtatgataagttcaagggtgccccgtatgtttttggaaaatgggagtataatggaaataaggaatccgatcttgcctgatcaatcctga

SEQ ID NO: 2 - protein, 389 aa, Bacillus licheniformis
  CDS alr
MSLKPFYRKTWAEIDLTALKENVRNMKRHIGEHVRLMAVVKANAYGHGDAQVAKAALAEGASILAVALLDEALSLRAQGIEEPILVLGAVPTEYASIAAEKRIIVTGYSVGWLKDVLGFLNEAEAPLEYHLKIDTGMGRLGCKTEEEIKEMMEMTESNDKLNCTGVFTHFATADEKDTDYFNMQLDRFKELISPLPLDRLMVHSSNSAAGLRFREQLFNAVRFGIGMYGLAPSTEIKDELPFRLREVFSLHTELTHVKKIKKGESVSYGATYTAQRDEWIGTVPVGYADGWLRRLAGTEVLIDGKRQKIAGRICMDQFMISLAEEYPVGTKVTLIGKQKDEWISVDEIAQNLQTINYEITCMISSRVPRMFLENGSIMEIRNPILPDQS

SEQ ID NO: 3 - DNA, 1170 bp, Bacillus subtilis
  CDS alrA
atgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaa

SEQ ID NO: 4 - protein, 389 aa, Bacillus subtilis
  CDS alrA
MSTKPFYRDTWAEIDLSAIKENVSNMKKHIGEHVHLMAVVKANAYGHGDAETAKAALDAGASCLAVAILDEAISLRKKGLKAPILVLGAVPPEYVAIAAEYDVTLTGYSVEWLQEAARHTKKGSLHFHLKVDTGMNRLGVKTEEEVQNVMAILDRNPRLKCKGVFTHFATADEKERGYFLMQFERFKELIAPLPLKNLMVHCANSAAGLRLKKGFFNAVRFGIGMYGLRPSADMSDEIPFQLRPAFTLHSTLSHVKLIRKGESVSYGAEYTAEKDTWIGTVPVGYADGWLRKLKGTDILVKGKRLKIAGRICMDQFMVELDQEYPPGTKVTLIGRQGDEYISMDEIAGRLETINYEVACTISSRVPRMFLENGSIMEVRNPLLQVNISN

SEQ ID NO: 5 - DNA, 1254 bp, Bacillus subtilis
  functional fragment alrA
ttttcgttagacatcgtttccctttagcctttaattttagtatgatatgtaaatgatattgaataaaagctaggaagtgtcgtaatgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaa

SEQ ID NO: 6 - DNA, 32 bp, Artificial Sequence
  Oligonucleotide PCR-amplification of pE194
tatatacagctggattcacaaaaaataggcac

SEQ ID NO: 7 - DNA, 33 bp, Artificial Sequence
  Oligonucleotide PCR-amplification of pE194
tatatacagctggattatgtcttttgcgcagtc

SEQ ID NO: 8 - DNA, 30 bp, Artificial Sequence
  Oligonucleotide PCR-amplification of functional mRFP cassette
tatatggatccgtaatcagggtatcgaggc

SEQ ID NO: 9 - DNA, 31 bp, Artificial Sequence
  Oligonucleotide PCR-amplification of functional mRFP cassette
tatatggatccctcattaggcgggctactaa

SEQ ID NO: 10 - DNA, 45 bp, Artificial Sequence
  Oligonucleotide Quickchange G to C, BsaI site removal
caagacaaaaagtggaaaagtcagaccatggagagaaaagaaaat

SEQ ID NO: 11 - DNA, 45 bp, Artificial Sequence
  Oligonucleotide Quickchange G to C, BsaI site removal
attttcttttctctccatggtctgacttttccactttttgtcttg

SEQ ID NO: 12 - DNA, 47 bp, Artificial Sequence
  Oligonucleotide Quickchange G to C, BpiI site removal
aaaaggattgaaggatgcttaggtagacgagttattaatagctgaat

SEQ ID NO: 13 - DNA, 47 bp, Artificial Sequence
  Oligonucleotide Quickchange G to C, BpiI site removal
attcagctattaataactcgtctacctaagcatccttcaatcctttt

SEQ ID NO: 14 - DNA, 1015 bp, Artificial Sequence
  functional fragment Type-II mRFP cassette
acatatgtcgagggtcttcggtaccttgtcggataaagctgtgttatattatgtcttggtgttaaatacacacgcttaacgatttatgcagagggtgctgcaggcggcagttctgtacaaaaatgacctaagcggaggaaaaaaaccattatattaggaggaaataacatggcctcttcagaggatgttattaaagaatttatgcggtttaaggtgaggatggaaggctcggtgaacggacatgagttcgaaattgagggagaaggtgaaggccgcccttatgaaggtactcagacagcgaaattgaaagtcacgaaaggcggaccgctgccgtttgcttgggacattctctcacctcaatttcaatatggctcaaaagcctacgtaaaacacccggctgacatccctgattacttaaagctatccttcccggagggctttaaatgggaacgagttatgaattttgaggacggcggcgtcgttactgtcacacaggattcttcccttcaggatggcgaatttatttacaaagtaaaacttcgtggaactaacttcccaagcgatggtcccgtgatgcaaaaaaaaacaatgggatgggaagcatctacggaacgtatgtatccggaggatggagccttaaagggtgaaatcaaaatgcgcctgaaacttaaagatggcggacactatgacgcggaagttaaaacaacatatatggctaaaaaaccagtccaactgccgggagcatataagacggatataaagttggacattaccagccataatgaagattacacgattgtggaacagtatgagagagcagagggcagacatagcacaggcgcgtaagaattaatgaaaaataagcggcagcctgcttttccatgcgggctgccgcttatcgggttattgcggccgcgtttataatgaagaccctagccatattctaacaaatagcatatagaaaaagctagtgtttttagcactagctttttcttcattctgatgaagggagctc

SEQ ID NO: 15 - DNA, 36 bp, Artificial Sequence
  Oligonucleotide PCR-amplification of functional pUK57S
tattagaattcaaatctttttgttccattaaagggc

SEQ ID NO: 16 - DNA, 35 bp, Artificial Sequence
  Oligonucleotide PCR-amplification of functional pUK57S
tatagaattcgccactacgtaaccaacatgattaa

SEQ ID NO: 17 - DNA, 35 bp, Artificial Sequence
  Oligonucleotide PCR-amplification of functional alrA fragment
tataggaattcttttcgttagacatcgtttccctt

SEQ ID NO: 18 - DNA, 36 bp, Artificial Sequence
  Oligonucleotide PCR-amplification of functional alrA fragment
tatagaattcttaattgcttatatttacctgcaata

SEQ ID NO: 19 - DNA, 35 bp, Artificial Sequence
  Oligonucleotide PCR-amplification of functional pUK57S
atatagaattccgtctacctaagcatccttcaatc

SEQ ID NO: 20 - DNA, 37 bp, Artificial Sequence
  Oligonucleotide PCR-amplification of promoter PaprE
tatatgaagaccttcgactcgggacctctttccctcg

SEQ ID NO: 21 - DNA, 41 bp, Artificial Sequence
  Oligonucleotide PCR-amplification of promoter PaprE
tatagaagacttatatctcactctcctcctctttattcaga

SEQ ID NO: 22 - DNA, 28 bp, Artificial Sequence
  Oligonucleotide PCR-amplification of protease pUK56
tataatgatgaggaaaaagagtttttgg

SEQ ID NO: 23 - DNA, 36 bp, Artificial Sequence
  Oligonucleotide PCR-amplification of protease pUK56
tatagaagacttgctattagcgtgttgccgcttctg

SEQ ID NO: 24 - DNA, 1182 bp, Bacillus licheniformis
  CDS yncD
atgaaaaagctttgccgtgaagtttgggtagaggtaaatcttgatgcgatcaaaaaaaatttgcgcgcgtttcggcggcatattccgaaaaagagcaaaattatggctgtcgtaaaagcgaatgcttatggtcacggatcggtggaagttgcacgccatgcacttgaacatggtgcgagtgagctcgccgttgcctcggtggaggaagcggtcgttttacgaaaagcggggattaaagcgccgatccttgtgcttggtttcaccccgctgagctgtgtgaaagaagcggcagcttggaatatatcgttatcagcttttcaagttgactggattaaagaagcgaacgagatattggaaaatgaagcagatcctaaccggctggctgttcatatcaatgtggataccggcatggggcgtttaggtgtacgaacaaaggaaaagcttttagcaatcgtggaagcgctgacggcaagtgaaaacctcgaatgggaaggaatttttacgcatttttccacagctgacgaaccggatactgagctaaccatgattcaacacgaaaagtttatcagctttcttcgctttctgaaagaacaaggctttaagctgcctacggtgcatatgaacaatacggccgcggcgatcgcttttccggaattcagcgctgatatgattcgcttaggcatcggaatgtatggattatatccttccgattatatcaggcagcttaatctcgttaagcttgtgcctgcactaagcttgaaggcgcgaatcgcttatgtgaaaaccatgttgactgaaccgcggacggttagttatggtgctacatatgttgcagagcgcggggaagtcattgccacaattccggtcggctatgctgacggctattcccgtgaactttccaaccgcggttttattcttcatcgaggaagacgagtgccggtggcgggaagagtaacaatggatatgataatggtcagtctgggagagggtgaaggtaaacaaggagaggaagtcgtgatttacggccggcaaaagggagcagagatatctgttgatgaaattgcggaaatgcttgatacgatcaactatgaagtggtatctaccataagctggcgcgtccctcgtttttatataagagacggcgagatttttaaaaagtcgaccccgctgttatacgtgtag

SEQ ID NO: 25 - protein, 393 aa, Bacillus licheniformis
  CDS yncD
MKKLCREVWVEVNLDAIKKNLRAFRRHIPKKSKIMAVVKANAYGHGSVEVARHALEHGASELAVASVEEAVVLRKAGIKAPILVLGFTPLSCVKEAAAWNISLSAFQVDWIKEANEILENEADPNRLAVHINVDTGMGRLGVRTKEKLLAIVEALTASENLEWEGIFTHFSTADEPDTELTMIQHEKFISFLRFLKEQGFKLPTVHMNNTAAAIAFPEFSADMIRLGIGMYGLYPSDYIRQLNLVKLVPALSLKARIAYVKTMLTEPRTVSYGATYVAERGEVIATIPVGYADGYSRELSNRGFILHRGRRVPVAGRVTMDMIMVSLGEGEGKQGEEVVIYGRQKGAEISVDEIAEMLDTINYEVVSTISWRVPRFYIRDGEIFKKSTPLLYV

SEQ ID NO: 26 - DNA, 1027 bp, Artificial Sequence
  homology regions Fusion of 5-prime and 3-prime regions of aprE gene with flanking BsaI sites
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 27 - DNA, 21 bp, Artificial Sequence
  Oligonucleotide PCR-amplification of aprE genomic region
ccggttgtcattgatccttta

SEQ ID NO: 28 - DNA, 21 bp, Artificial Sequence
  Oligonucleotide PCR-amplification of aprE genomic region
atcctcctgcaaaaaccgtat

SEQ ID NO: 29 - DNA, 1022 bp, Artificial Sequence
  homology regions Fusion of 5-prime and 3-prime regions of amyB gene with flanking BsaI sites
ggtctcgacccacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgtccgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtatttgttaaaaattcggaatatttatacaatatcatatgtttcacattgaaaggggaggagaatctagaagagcagagaggacggatttcctgaaggaaatccgtttttttattttgcccgtcttataaatttctttgattacattttataattaattttaacaaagtgtcatcagccctcaggaaggacttgctgacagtttgaatcgcataggtaaggcggggatgaaatggcaacgttatctgatgtagcaaagaaagcaaatgtgtcgaaaatgacggtatcgcgggtgatcaatcatcctgagactgtgacggatgaattgaaaaagcttgttcattccgcaatgaaggagctcaattatataccgaactatgcagcaagagcgctcgttcaaaacagaacacaggtcgtcaagctgctcatactggaagaaatggatacaacagaaccttattatatgaatctgttaacgggaatcagccgcgagctggaccgtcatcattatgctttgcagcttgtcacaaggaaatctctcaatatcggccagtgcgacggcattatctcaggagacc

SEQ ID NO: 30 - DNA, 22 bp, Artificial Sequence
  Oligonucleotide PCR-amplification of amyB genomic region
agaagcatgaagggcatgcgac

SEQ ID NO: 31 - DNA, 21 bp, Artificial Sequence
  Oligonucleotide PCR-amplification of amyB genomic region
caacaacaggctgtctgacgg

SEQ ID NO: 32 - DNA, 1022 bp, Artificial Sequence
  homology regions Fusion of 5-prime and 3-prime regions of sigF gene with flanking BsaI sites
ggtctcgacccaaaatcattcgccttgagcaatcagagcagcgtgcacttgaaacgttgggggtggcgtcatgaaaaatgaaatgaacattcagtttacagcgctcagccaaaatgaatcgtttgcacgggtgacagtcgctgcttttatcgctcagcttgacccgacgatggatgaactgaccgaaattaaaacggtcgtatccgaagcggtcacaaacgcgatcattcacggttatgaaaactcagggcagggaaacgtatatatttccgtcactctcgaggaccatattgtctatttaacgatccgcgacgaaggagtcggcatccctgatcttgaagaagcgcgccagcccctgttcacgacaaagcctgaactcgagcggtcgggaatgggctttacgatcatggaaaatttcatggatgatatttcgatcgactcctcacctgagatgggaaccacaatacacttaacaaagcacttatcaaaaagcaaagcgctttgcaattaatgaggctgctcatgttgcaggcagcctcggatgtccgatgaaaaaccggacgctcttgggagcgttccggttttttttgtgtggtaatttatggtcttttgcgcctttctgaatgataaatgggatgtacttcatactacaactataaccatcatataggaagtgacccagatggaacgtcaagtttttatcagactccgccaccggcttgaggcagatccggatgaattgattttgctcggccatatcgcacaggtagcgggtgaccgcggatacaaagaaaagcttgaacggctgcctatttatcaggtcagcaaagcggatcaaagcatggtggtgctggacgtgatgaaggtgattgaagctgtacataaatcgtttcctgaccttgatgtccaaaccgtcggcggttctgaaaccatcgtggagatccaatatccgaaaaggggtctgtcgcccgtgcttttcatcgccgtctggctgctcttgttcgtcggtgcctcaggagacc

SEQ ID NO: 33 - DNA, 19 bp, Artificial Sequence
  Oligonucleotide PCR-amplification of sigF genomic region
agcagctcggcggagaaat

SEQ ID NO: 34 - DNA, 21 bp, Artificial Sequence
  Oligonucleotide PCR-amplification of sigF genomic region
accttgcccgtcaccatttcg

SEQ ID NO: 35 - DNA, 1363 bp, Artificial Sequence
  homology regions Fusion of 5-prime and 3-prime regions of pga genes with flanking BsaI sites
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 36 - DNA, 20 bp, Artificial Sequence
  Oligonucleotide PCR-amplification of pga genomic region
aaagccttctcctctctatt

SEQ ID NO: 37 - DNA, 20 bp, Artificial Sequence
  Oligonucleotide PCR-amplification of pga genomic region
ttcttgaaaaagacaaggtc

SEQ ID NO: 38 - DNA, 1034 bp, Artificial Sequence
  homology regions Fusion of 5-prime and 3-prime regions of alr gene with flanking BsaI sites
ggtctcgacccggatcccttgaagagaaagagatggcgactgaagacggcaaacggatcgtcatcacatatggcggtgaaaaatcctttacattgattcaggaaaaagcacgcgtcgccaaaacatccacttccgtatccatgaacggagagcccgttgacctcggcttcacggtcggcgcactgacggataaatcgttgtcatggacatatgacggagtcgattacttcatctcatcagaagatctttctcaagatgaacttctgatggttgcaaaaagcatgcagggacagtcttcaaaatagactgtgccgtacccggcagcctgttttccgcccggaagcggaaagcaggcttttttatatttgcgtcgcaagcgtatgatttcgacagcttttccgtaaaatgcataccgtgccagcaatttttcttttgttcagggctgatgatcccgtgcaaaatttccctttctccgaactttttagtatgatgggaaggacgagtgaaacaaggaacaaaattgatgaattagcggaaaaacaactttgcttgcgaaaagaataatgatatgattatgaatggaatggatagagtgttgtatccgtaagtttggtggaggtgtatgtttttgtctgaatccagcgcaacaactgaaatattgattcgcttgccagaagctttagtatcagaactggatggtgtcgtcatgcaagataacgggagcagaaatgaactgatttaccaagccacaaaaatgtatttacgcgaacgcaaaaatcgacaaattcgggaatcgatgagacgcggttatatggagatggccaagatcaatttgaacatctcttctgaggctcaatttgcagagtatgaggctgaaaacacagtagagcgcttagtaagcggaggataatcatttgattgttaaacgcggcgatgtttattttgctgacctatctcctgttgttggctcagaacaaggcggggtgcgcccggttttagtgattcaaaacaacatcgccatggctcaggagacc

SEQ ID NO: 39 - DNA, 25 bp, Artificial Sequence
  Oligonucleotide PCR-amplification of alr genomic region
gatggtaaaggttgagtttacaagc

SEQ ID NO: 40 - DNA, 25 bp, Artificial Sequence
  Oligonucleotide PCR-amplification of alr genomic region
catttcatcatcgagatgggtgatt

SEQ ID NO: 41 - DNA, 1020 bp, Artificial Sequence
  homology regions Fusion of 5-prime and 3-prime regions of yncD gene with flanking BsaI sites
ggtctcgaccctaactgcatatgtgtgtctgaaaaagaaaactttaaattagggattgctccggcatctgtttttggaggaacgaacatgtcgggattctctaaatcccgcatcacgtcgcgggggccaaaatcggtcgcgcccgcaccatcgtttcttattggatgcggcacgtgattgtaccaagctggagtcccggctgttcttttcatgcggaaagctcccttcacaggtcgtgtataccgaaatttatgacagggacgcttaaaatatatcaaatcggagccttattaagacacttttaaaaaactagatggacagatttgcatttcctttggcagcagaaggaaaccttcgatttaaagtctgcacggcacgggctcctgctgtttgccttccaccgctaacgattactggctgaatgatttcgcccgaagctgaataagaatgagatagaaagcatccgggcaattttcgaacggctacatgaaatgaatactatgggacaagatagttttcccatactcacaggccgtttgctcgatcattgatcaaaataaaaaagagcaatttcttgacaaaaaaatttatttatgatataatttaataattaaatattggtgtgtataataggattctcctggccgggagaatcctatttttttgcgtttatgaaggagtgtccgacatgaaaaaagatgaatcgagtataacgtcccttatttcggcttttggcagagcttatcacagccagtatgatgaaccaaaaatctttgatgataatctggccaaatcgctcatttcccaacaagaatttgctgatatcaaaaacagcttcattcaaggcatacagttcttcaacaaagctatagcggaaaaatacgagggagacgatgaagaaatattaagatggatcacacaagttcagctctctccgacgccgctcgcccgtgccgcttattgcgaaagagtgctgcatcatgagattcggcttgggacagaacaatacctcaggagacc

SEQ ID NO: 42 - DNA, 25 bp, Artificial Sequence
  Oligonucleotide PCR-amplification of yncD genomic region
atcctcaagcccttgaatcgagtgc

SEQ ID NO: 43 - DNA, 25 bp, Artificial Sequence
  Oligonucleotide PCR-amplification of yncD genomic region
ctttttcaatttctgtgtcgctggg

SEQ ID NO: 44 - DNA, 1185 bp, Bacillus subtilis
  CDS yncD
atgataaagctttgccgagaagtttggatcgaagtcaatcttgatgccgttaaaaagaatttgcgagcgatccgccgtcatattccacataagagcaaaattatggctgttgtaaaagcgaacggttatggccacgggtctatagaagtggcacgccatgcactggaacacggggcgagtgagctcgctgttgccagtgtggaggaaggaatcgttttacgtaaagctgggattacagcacctatccttgtgcttggttttacatcccttagttgtgtaaagaagtcagcagcttggaatataacattatcagcatttcaggttgattggatgaaagaagcaaacgagatattggaaaaagaagcgagtgctaaccggctggccattcatattaacgtggataccggcatgggacgattaggtgtacgcacaaaggaagaacttttagaggtagtgaaagccttaaaggcaagtaaattcctcagatggacagggattttcacacatttttcgacagctgatgaaccggacacaacactcacaaagctgcagcatgaaaaattcatcagttttctcagctttttaaaaaaacaggggattgagctgcccaccgtgcatatgtgcaacacggctgcagctatcgcatttccggaatttagcgctgatatgatccgtttaggtatcggcttatatggtttatatccttcagcctatattaaacaactaaatctcgttaagcttgaacctgcgttaagtttaaaggcacgcatcgcttatgtgaaaaccatgcggacagaaccccgaaccgttagctatggtgctacatacatcgcagaacctaatgaagtaattgctacactcccgattggctatgctgacggctattctcgtgccctttccaatcgcgggtttgttcttcatcgcggaaaacgagtgccagtggcgggaagagtaacaatggatatgatcatggtcagtttaggagaaaacggtgaaggtaaacaaggagatgaagtcgtgatttacggtaagcaaaaaggagccgagatttccgttgatgaagtggctgaaatgctcaatacgattaactatgaagtcgtgtctacattaagccgccgcatccctcgtttttatataagagatggcgagatttttaaagtatcaactccagtattatacgtgtag

SEQ ID NO: 45 - protein, 394 aa, Bacillus subtilis [sequence omitted]

SEQ ID NO: 46 - DNA, 84 bp, Bacillus subtilis
  promoter alrA
ttttcgttagacatcgtttccctttagcctttaattttagtatgatatgtaaatgatattgaataaaagctaggaagtgtcgta

SEQ ID NO: 47 - protein, 392 aa, Bacillus pumilus
  CDS alr
MDTNAFYRDTWAEINLSAIKHNVSHMKDHIGQSVQLMAVVKANAYGHGDIEVASAALKAGADILAVAFLDEAVSLRNKGIKAPILVLGAVPTEYVKVAVRYNVIMTAYSIDWLKDVINMMEGQMGQPIRFHLKIDSGMNRLGVKTIEQVNEVKKLVDEHSYLQLEGVFTHFATADEKDEAYFDRQVDTFQTLLEPLHTDKLLIHCANSAAGLRFKDVLFNMVRFGISMYGLSPSEEIKDELPFKLEEAMSLHTKLAHVKHIQKGESVSYGATYTANQDTWIGTVPVGYADGWIRKLAGTEVLVGGKRRKITGRICMDQFMVELKDKLPVGEPVVLIGKQGEDMIQVDEIAKRLETINYEITCSIGHRVPRVYIEDGKRVQVRNPLLQGGPSF

SEQ ID NO: 48 - protein, 389 aa, Bacillus velezensis
  CDS alr
MNTASFYRETWAEINLSAIKENVTHMKKHIGQNVHLMAVVKANAYGHGDLEVGKAALEAGASCLAVAILDEAISLRKRGITAPILVLGAVPPEYVQAAAEYDVTLTGYSVEWLQEAARHLGSATVPFHLKVDTGMNRLGVKTEEEIQSVLKILSQNPGLVCKGVFTHFATADEKNRDYFLFQFDRFKTLIAPLPLKELMVHCANSAAGLRLKKGFFNAVRFGISMYGLRPSADIQSEIPFQLKPAFALHSVLSHVKKIRKGESVSYGATYTAEKDQWIGTVPIGYADGWLRKLSGTSVLIGGKRMNIAGRICMDQLMVELDQSYPPGTKVTLIGSQKEETITMDEIAGRLGTINYEVPCTISSRVPRMFLENESIMEVRNPLLQDNTSK

SEQ ID NO: 49 - protein, 389 aa, Bacillus atrophaeus
  CDS alr
MNIKPFYRDTWAEINLSAIEENVSNMKKHIGDRVELMAVVKANAYGHGDAEVAEAALSAGASCLAVAILDEAISLRKRGLAAPILVLGAVPPEYVEIAAEYDVTLTGYSVEWIQEAARHIKETPVHFHLKVDTGMNRLGVKTEEEVQNVMKILERNPGLLCKGVFTHFATADEKDRSYFLMQFERFKELIAPLPWKQLMVHCANSAAGLRLKKGFFNAVRFGVGMYGLRPSAEITNEIPFRLHQAFTLNSTLTHVKKIHKGESVSYGATYTAEKDQWIGTVPVGYADGWLRKLQGTEVLINGKRLKIAGRICMDQFMVELDKEYPPGSRVTLIGRQKDDCITVDEIASRLETINYEVTCTISSRVPRMFLKNGSIMEVRNPLLQENASK

SEQ ID NO: 50 - protein, 389 aa, Bacillus mojavensis
  CDS alr
MSTKPFYRDTWAEIDLSAIKENVSNMKKHIGEQVHLMAVVKANAYGHGDAEVAKAALEAGASCLAVAILDEAISLRQRGLTAPILVLGAVPPEYVKIAAEYDVTLTGYSVEWLQEAARHLKKASLHFHLKVDTGMNRLGVKTEEEIHHVMTILERNPGLKCKGVFTHFATADEKQRGYFLMQFERFKELIAPLPLKKLMVHCANSAAGLRLKKGFFNAVRFGIGMYGLRPSAEITDEIPFKLRPAFTLHSTLSHVKKVRKGESVSYGAEYTAKKDTWIGTVPVGYADGWLRKLKGTEILVKGKRLKIAGRICMDQFMVELDHEYPPGTKVTLIGRQGDEYISMDEIAGRLETINYEVACTISSRVPRMFLENGSIMEVRNPLLQGNASK

SEQ ID NO: 51 - protein, 392 aa, Bacillus xiamenensis
  CDS alr
MDTNAFYRDTWAEIDLTAIKHNVSHMKDHIGQNVQLMAVVKANAYGHGDIEVARAALKAGADLLAVAFLDEAISLRNKGIKAPILVLGAVPAEYVKVAVKYNVIMTAYSIEWLREVVKVVQGQMGQPIRFHLKMDSGMNRLGVKTLEQVNEVKELVREHSYLKLEGVFTHFATADEQDEDYFNRQVDTFHTLLQPLRADQLLIHCANSAAGLRFKEVLFNMVRFGISMYGLSPSEEIKDELPFKLEEALSLHTKLAHVKLIQKGESVSYGATYTANQDTWIGTVPVGYADGWIRKLAGTEVLVGGKRRKIAGRVCMDQFMVELKEHMPAGEPVVLIGAQGAEKVSVDEIAKRLETINYEVTCSIGHRVPRVYIEDGKRVYVRNPLLQDGPNF

SEQ ID NO: 52 - protein, 393 aa, Bacillus zhangzhouensis
  CDS alr
MDTNAFYRDTWAEIDLTAIKHNVSHMKDHIGQNVQLMAVVKANAYGHGDIEVARAALKAGADLLAVAFLDEAISLRNKGIKAPILVLGAVPPEYVKVAVRYNVIMTAYSIEWLRDVVKVVKGQIGQPFRFHLKIDSGMNRLGVKTLAQVNEVKELSRDHSYLHLEGVFTHFATADEKDEDYFDMQVDTFQTLLQPLHTDKLLVHCANSAAGLRFKEVLFNMVRFGISMYGLSPSEEIKDELPFKLEEAMSLHTKLAHVKLIRKGESVSYGATYTANQDTWIGTVPVGYADGWIRKLAGTEVLVGGKRRKIAGRVCMDQFMVELKEDIPAGEPVVLIGSQGEEKVSVDEIAKRLETINYEVTCSIGHRVPRVYKEDGKRVHVRNPLLQGGPSFL

SEQ ID NO: 53 - protein, 391 aa, Bacillus sonorensis
  CDS alr
MSLKPFYRKTWAEIDLTAVKENVRNMKRHIGGRVHLMAVVKANAYGHGDAQVAKAALEEGASILAVAFLDEALSLRGQGIDAPILVLGAVPPEYVGIAAEHRITVTAYSVRWLKDVLGFIGSVSVPLDFHLKIDTGMGRLGCKTEEEIKEMMDMTEASDKLNCRGVFTHFATADEKETDYFNMQLDRFKELIKPLPLDRLMVHSSNSAAGMRFKEQLFNAVRFGVSMYGLSPSSEIKDELPFPLKEVFSLHTELVHVKKIHKGESVSYGATYTAEHDEWIGTVPVGYADGWLRRLAGSEVLIDGKRQKIAGRICMDQFMISLSEEYPVGTKVTLIGKQKDESISVDEIAKKLQTINYEITCMISSRVPRMFLEKGSIMEIRNPILPNDIEK

SEQ ID NO: 54 - protein, 393 aa, Bacillus pumilus
  CDS yncD
MKKLCREVWIEVNLDAIKRNIEAIQAHIPRKSKIMAVVKANAYGHGSVEVARQALESGATELAVASLEEGIVLRRAKIEAPILVLGFTPLDCVKRAAAWRIDLSGLREDWIVEANEILAEEESQRRLGIHVNVDTGMGRLGVRTKEELLGVVEALEKSENLRWDGIFTHFSTADEPDPDFTLLQHSIFIDFLRFLKKQGIKLPTVHMNNTAAAIAFPEFSADMIRLGIGLYGLYPSQYIESLDAVQLEPALSLKARIAFVKEMVTEPRTVSYGATYVAKRDEVIATIPIGYADGYSRALSNRGFMLFRGERMPIAGRVTMDMTMISLGEMKAKQGEEVVIYGRQKGGEISVDEIAEMLNTINYEVIATLSRRVPRFYRRGGKIIKISTPVMYV

SEQ ID NO: 55 - protein, 393 aa, Bacillus velezensis
  CDS yncD
MKKLCREVWIEVDLDAIKKNVRAIRRHIPNTSKIMAVVKANAYGHGSVEVARQALESGASELAVASVEEGIVLRRAGITAPILVLGFTALSCVKKSAVWNITLSAFQVSWIKKANRILEKEADSKRLSIHINVDTGMGRLGVRTEEDLLAVVKALKSSTYLSWDGIFTHFSTADEPDTELTMLQHEKFISFLRFLKNQDITLPTVHMCNTAAAIAFPEFSADMIRLGIGLYGLYPSASIKELNLVDLTPALSLKARIAYVKAMVTDPRTVSYGATYIAEPGEIIATIPIGYGDGYSRALSNRGFILHRGRRVPVAGRVTMDMIMVSLGENEGKQGEEVVIYGKQQGAEISVDEIAEMLGTISYEVLSVLSRRVPRFYFRDGKIIKISAPVLYV

SEQ ID NO: 56 - protein, 393 aa, Bacillus atrophaeus
  CDS yncD
MTKLCREVWIEVNLDAIKKNLRAIRHHIPKKSKIMAVVKANAYGHGSVEVARHALENGASELAVASVEEGIVLRKAGITAPILVLGFTSLSCVKESAAWNIELSAFQVDWIKEANEILKNEARPNRLGIHINVDTGMGRLGVRTKEELLAVVNALTASKFLRWAGIFTHFSTADEPDTTLTKLQHDKFIRFLRFLKNQGIELPTVHMSNTAAAIAFPEFSADMIRLGIGLYGLYPSAYIKQLHLVKLEPALSLKARIAYVKTMRTEPRTISYGATYIAEPDEVIATLPVGYADGYSRALSNRGFVLHRGRRVPVVGRVTMDLIMVSLGESEGKQGEEVVIYGQQKGAEISVDEVAEMLNTINYEVVSTLSRRIPRFYIRDGGIFKVSTPVMYV

SEQ ID NO: 57 - protein, 362 aa, Bacillus mojavensis
  CDS yncD
MAVVKANGYGHGSVEVARHALEHGASELAVASVEEGIVLRKAGITAPILVLGFTSLSCVKKSAAWNIELSAFQVDWIKKANRILENEESTESTSRLGIHINVDTGMGRLGVRTKEKLLAVVEALTESKFLRWSGIFTHFSTADEADTTLTKLQHEKFINFLSFLKNRGIELPTVHMSNTAAAIAFPEYSADMIRLGIGLYGLYPSDYIKQLNLVKLEPALSLKARIAYVKTMRTEPRTISYGATYIAEPEEVIATLPVGYADGYSRALSNRGFVLHRGKRVPVVGRVTMDMIMVSLGESEGKQGEEVVIYGHQKGAEIPVDEVAEMLNTINYEVVSTLSRRVPRFYIRDGEMFKVSTPVMYV

SEQ ID NO: 58 - protein, 393 aa, Bacillus xiamenensis
  CDS yncD
MKNLCREVWIEVNLDAIKRNIEAIQAHIPRKSKIMAVVKANAYGHGSVEVARQALESGATELAVASLEEGIVLRRAKVEAPILVLGFTPLDCVKKAAAWKIDLSGLRADWIKQANEILEEEESQNQLGIHVNVDTGMGRLGVRTKEELLAVVEALEKSEKLRWDGIFTHFSSADEPDPDFTLLQHSIFIDFLRFLKKQGITLPTVHMNNTAAAIAFPEFSADMIRLGIGLYGLYPSTYIQSLDAVQLEPALSLKARIAFVKEMVTEPRTVSYGATYVAKPDEVIATIPIGYADGYSRALSNRGFMLFCGERVPIAGRVTMDMTMISLGDMKAKQGEEVVIYGRQKGGEISVDEIAEMLNTINYEVVATLSRRVPRFYRRGGQMIKISTPVMYV

SEQ ID NO: 59 - protein, 393 aa, Bacillus zhangzhouensis
  CDS yncD
MIKLCREVWIEVNLDAIKRNIEAIQAHIPRKSKIMAVVKANAYGHGSVEVARQALESGATELAVASLEEGIVLRRAKIEAPILVLGFTPLDCVKKAAAWRIDLSGLRADWINQANAILAEEESQHRLGIHVNVDTGMGRLGVRTKEELLEVVEALEESEYLRWDGIFTHFSTADEPDPDFTLLQHSIFIDFLRFLKKQGIPLPTVHMNNTAASIAFPEFSADMIRLGIGLYGLYPSQYIESLDAVQLEPALSLKARIAFVKEMVTKPRTVSYGATYVAKPDEVIATIPIGYADGYSRALSNRGFMLFRGKRVPIAGRVTMDMTMISLGEMKAKQGEEVVIYGRQKGGDISVDEIAEMLNTINYEVIATLSRRVPRFYRRGGKIIKISTPVMYV

SEQ ID NO: 60 - protein, 393 aa, Bacillus sonorensis
  CDS yncD
MKKLCREVWAEVDLDAIKKNLRAIRHHIPKKSKIMAVVKANAYGHGSVEVARHALEHGASELAVASLEEGIVLRKAGITAPILVLGFTPLRCVKESAAWNITLSAFQVDWIKEANEILEKEAETNRLNIHINVDTGMGRLGVRTKEKLLEIVKALTASKFLRWKGIFTHFSTADEPDTTLTRLQHDKFISFLRFLKDQGFELPTVHMNNTAASIAFPEFSADMIRLGIGMYGLYPSEYIKQLNLVKLVPALSLKARIAYVKTMLTEPRTISYGATYIAEPGEVIATLPVGYADGYSRALSNRGFVLHRGRRVPVAGRVTMDMIMVSLGEGEGRQGEEVVIYGRQKGAEISVDEVADMLDTINYEVVSTISRRVPRIYIRDGEILKVSTPVLYV

(19) United States
(12) Patent Application Publication
(10) Pub. No.: US 2023/0272358 A1
(43) Pub. Date: Aug. 31, 2023

(54) ALANINE RACEMASE SINGLE DELETION AND TRANSCOMPLEMENTATION

(71) Applicant: BASF SE, Ludwigshafen (DE)

(72) Inventors: Stefan Jenewein, Ludwigshafen (DE); Max Fabian Felle, Ludwigshafen (DE); Christopher Sauer, Ludwigshafen (DE); Tobias Klein, Ludwigshafen (DE)

(21) Appl. No.: 18/017,426

(22) PCT Filed: Jul. 23, 2021
(86) PCT No.: PCT/EP2021/070662

§ 371 Date: Jan. 23, 2023

(30) Foreign Application Priority Data
Jul. 24, 2020 (EP) 20187745.3
Jul. 24, 2020 (EP) 20187746.1

(51) Int. Cl. C12N 9/48; C12N 15/75; C12N 9/90; C12N 1/20

(52) U.S. Cl. CPC C12N 9/48; C12N 15/75; C12N 9/90; C12N 1/20; C12R 2001/10

(57) ABSTRACT

The present invention relates to a Bacillus host cell belonging to the species Bacillus lichemformis or Bacillus pumilus in which the chromosomal alr gene has been inactivated. Said bacterial host cell comprises a plasmid comprising at least one autonomous replication sequence, a first polynucleotide encoding at least one polypeptide of interest, wherein said first polynucleotide is operably linked to a promoter, and a second polynucleotide encoding an alanine racemase which is not native to the host cell, wherein said second polynucleotide is operably linked to a promoter. The present invention further relates to a method for producing at least one polypeptide of interest based on cultivating the bacterial host cell of the present invention.

### FIELD OF THE INVENTION

The present invention relates to a Bacillus host cell belonging to the species Bacillus licheniformis or Bacillus pumilus in which the chromosomal alr gene has been inactivated. Said bacterial host cell comprises a plasmid comprising at least one autonomous replication sequence, a first polynucleotide encoding at least one polypeptide of interest, wherein said first polynucleotide is operably linked to a promoter, and a second polynucleotide encoding an alanine racemase which is not native to the host cell, wherein said second polynucleotide is operably linked to a promoter. The present invention further relates to a method for producing at least one polypeptide of interest based on cultivating the bacterial host cell of the present invention.

### BACKGROUND OF THE INVENTION

Advances in genetic engineering techniques have allowed the improvement of microbial cells as producers of heterologous proteins. Protein production is typically achieved by the manipulation of gene expression in a microorganism such that it expresses large amounts of a recombinant protein.

Microorganisms of the Bacillus genus are widely applied as industrial workhorses for the production of valuable compounds, e.g. chemicals, polymers, proteins and in particular proteins like washing- and/or cleaning-active enzymes. The biotechnological production of these useful sub-stances is conducted via fermentation and subsequent purification of the product. Bacillus species are capable of secreting significant amounts of protein to the fermentation broth. This a1-lows a simple product purification process compared to intracellular production and explains the success of Bacillus host cells, such as B. licheniformis or B pumilus (see e.g. Kuppers et al., Microb Cell Fact. 2014; 13(1):46, or Schallmey et al., Can J Microbiol. 2004; 50(1):1-17) in industrial application.

For high-level production of compounds by recombinant production hosts stable expression systems are essential. Recombinant production hosts are genetically modified compared to the native wild-type hosts to produce the compound of interest at higher levels. However, recombinant production hosts have the disadvantage of lower fitness compared to wild-type hosts leading to outgrowth of wild-type cells in fermentation processes and loss of product yields.

Autonomous replicating plasmids are circular DNA plasmids that replicate independently from the host genome. Plasmids have been used in prokaryotes and eukaryotes for decades in biotechnological application for the production of compounds of interest.

Unlike some naturally occurring plasmids, most recombinant plasmids are rather unstable in bacteria—in particular when production of a compound of interest exerts a disadvantage for the fitness of the cell. Moreover, the stable maintenance of a plasmid is a metabolic burden to the bacterial host.

A number of approaches to maintain plasmids and therefore productivity of recombinant hosts have been tried. Positive selection conferred by, e.g., antibiotic resistance markers and auxotrophic resistance markers has been used to retain production yield at satisfactory level.

The use of antibiotic resistance markers on the plasmid and supplementing the media with antibiotics has been widely used as positive selection in fermentation processes. However, under conditions of strong production of a compound of interest, loss of plasmids within the cell population have been observed since the concentration of the antibiotic used for the plasmid selection often decreases during long-term cultivation as a result of dilution and/or enzymatic degradation. Moreover, the presence of antibiotics is generally not accepted in the final product and waste-water and, therefore, requires additional purification.

Auxotrophic markers, e.g. enzymes of the amino acid biosynthesis routes, can also be used for positive selection on a plasmid when pure and defined media is used for fermentation processes with host cells defective in the corresponding genes. Providing the auxotrophic marker on a multi-copy plasmid can exert a negative impact on cell growth and productivity of the cell as the enzymatic function is not balanced to cellular physiology compared with the wild-type host. Furthermore, cell lysis during fermentation processes can lead to cross-feeding of the compound made by the auxotrophic marker, rendering the system less effective for plasmid maintenance.

EP 3 083 965 A1 discloses a method for deletion of antibiotic resistance and/or creation of a plasmid stabilization in a host cell by deleting the chromosomal copy of the essential, cytoplasmatic gene frr (ribosome recycling factor) and placing it onto the plasmid. As a result, only plasmid-carrying cells can grow, making the host cell totally dependent on the plasmid. Moreover, cross-feeding effects as outlined for auxotrophic markers do not exist as full proteins cannot not be imported into the cell.

The disadvantage for construction of recombinant host cells is that deletion of the chromosomal gene can only be made in the presence of at least one gene copy on a plasmid. Replacement of such a plasmid with another plasmid, e.g. a plasmid that differs from the first plasmid by a different gene-of-interest intended for production, is tedious and might need a counterselection marker for efficient removal of the first plasmid.

As an alternative approach for protein production, the enzyme alanine racemase has been used for plasmid maintenance in prokaryotes. Alanine racemases (EC 5.1.1.1) are unique prokaryotic enzymes that convert L-alanine into D-alanine (Wasserman, S. A., E. Daub, P. Grisafi, D. Botstein, and C. T. Walsh. 1984. Catabolic alanine racemase from Salmonella typhimurium: DNA sequence, enzyme purification, and characterization. Biochemistry 23: 5182-5187). D-alanine is an essential component of the peptidoglycan layer that forms the basic component of the cell wall (Watanabe, A., T. Yoshimura, B. Mikami, H. Hayashi, H. Kagamiyama, and N. Esaki. 2002. Reaction mechanism of alanine racemase from Bacillus stearothermophilus: x-ray crystallographic studies of the enzyme bound with N-(5′-phosphopyridoxyl)alanine. J. Biol. Chem. 277: 19166-19172).

Ferrari et al. (Ferrari, E. 1985. Isolation of an alanine racemase gene from Bacillus subtilis and its use for plasmid maintenance in B. subtilis. Biotechnology 3:1003-1007) isolated the D-alanine racemase gene dal (also referred to as alr gene) of B. subtilis which led to rapid cell death upon deletion in B. subtilis and showed the effectiveness of the dal gene as selection marker when placed on a replicative plasmid in B. subtilis.

The alr gene of Lactobacillus plantarum was identified and its functionality as alanine racemase proven by complementation of the growth defect of E. coli defective in its two alanine racemase genes alr and dadX (P Hols, C Defrenne, T Ferain, S Derzelle, B Delplace, J Delcour Journal of Bacteriology Jun 1997, 179 (11) 3804-3807).

Similarly to the work of Ferrari et al., the alanine racemase genes of lactic acid bacteria (alr) from Lactococcus lactis and Lactobacillus plantarum were deleted on the genome and placed in trans on the plasmid which resulted in stable plasmid maintenance for 200 generations and showed the use of the homologous alr gene for application as food grade selection marker (Bron, P. A., M. G. Benchimol, J. Lambert, E. Palumbo, M. Deghorain, J. Delcour, W. M. de Vos, M. Kleerebezem, and P. Hols. 2002. Use of the alr gene as a food-grade selection marker in lactic acid bacteria. Appl. Environ. Microbiol. 68: 5663-5670.; Ferrari, 1985).

WO 2015/055558 describes the use of the Bacillus subtilis da/gene for plasmid maintenance in a B. subtilis host cell with an inactivated da/gene. The expression level of the dal gene on the plasmid was reduced by mutating the ribosome binding site RBS to a lower level compared to the unaltered RBS. Thereby, the plasmid copy number could be maintained at a high copy number and the amylase production yield increased.

In contrast to many gram-negative organisms such as Escherichia coli, Pseudomonas aeruginosa, and Salmonella typhimurium, most gram-positive bacteria investigated such as Bacillus stearothermophilus, Lactobacillus plantarum, and Corynebacterium glutamicum appeared to have only one alanine racemase gene (Pierce, K. J., S. P. Salifu, and M. Tangney. 2008. Gene cloning and characterization of a second alanine racemase from Bacillus subtilis encoded by yncD. FEMS Microbiol. Lett. 283: 69-74). For Bacillus subtilis a second alanine racemase gene, namely yncD, was identified and complementation with the yncD gene placed onto a plasmid in an D-alanine auxotrophic strain of E. coli shown (Pierce et al., 2008). Similarly, a second alanine racemase gene alr2 (homolog to B. subtilis yncD gene) was found in Bacillus licheniformis and it was shown that when expressed from a plasmid under the control of the lac promoter could complement the D-alanine auxotrophic phenotype of E. coli defective in two alanine racemase genes alr and dadX(Salifu, S. P., K. J. Pierce, and M. Tangney. 2008. Cloning and analysis of two alanine racemase genes from Bacillus licheniformis. Anals of Microbiology 58: 287-291).

A recent study (Munch, K. M., J. M uller, S. Wienecke, S. Bergmann, S. Heyber, R. Biedendieck, R. Munch, and D. Jahn. 2015. Polar Fixation of Plasmids during Recombinant Protein Production in Bacillus megaterium Results in Population Heterogeneity. Appl. Environ. Microbiol. 81: 5976-5986) describes the effects of cell heterogeneity on productivity of recombinant host cells during cultivation. Loss of productivity exemplified by heterologous protein production in Bacillus megaterium and B. subtilis was not caused by simple plasmid loss, however, by asymmetric distribution of plasmids during cell division leading to a small population of so called ‘high-producers’ and a large population of ‘low-producers’.

Therefore, it remains the need for stable plasmid-host systems leading to overall enhanced production of a compound.

### BRIEF SUMMARY OF THE INVENTION

It has been found in the studies underlying the present invention that the inactivation of the endogenous chromosomal alr gene in a Bacillus host cell and the introduction of a plasmid comprising a polynucleotide encoding an alanine racemase which is not native to the host cell, and a polynucleotide encoding at least one polypeptide of interest allows for increasing the expression of the polypeptide of interest as compared to a control cell. Specifically, the B. subtilis alrA gene was introduced into a host cell belonging to the species of B. licheniformis in which the endogenous alr gene has been inactivated. As B. pumilus, B. licheniformis comprises two endogenous genes encoding for an alanine racemase: alr and yncD. Interestingly, the expression enhancing effect was more pronounced when the alr gene was inactivated, as compared to when the yncD gene was inactivated (see Example 2).

Accordingly, the present invention relates to a method for producing at least one polypeptide of interest, said method comprising the steps of
a) providing a Bacillus host cell belonging to the species Bacillus licheniformis or Bacillus pumilus, in which the chromosomal alr gene has been inactivated and which comprises a plasmid comprising
   1. at least one autonomous replication sequence,
   2. a first polynucleotide encoding at least one polypeptide of interest, wherein said first polynucleotide is operably linked to a promoter, and
   3. a second polynucleotide encoding an alanine racemase which is not native to the host cell, wherein said second polynucleotide is operably linked to a promoter, and
b) cultivating the host cell under and conducive for maintaining said plasmid in said host cell and conducive for expressing said at least one polypeptide of interest, thereby producing said at least one polypeptide of interest.

In an embodiment of the method of the present invention, step a) comprises the following steps:
a1) providing a Bacillus host cell belonging to the species Bacillus licheniformis or Bacillus pumilus,
a2) inactivating the chromosomal alr gene of said host cell, and
a3) introducing into said host cell a plasmid comprising
   1. at least one autonomous replication sequence,
   2. a first polynucleotide encoding at least one polypeptide of interest, wherein said first polynucleotide is operably linked to a promoter, and
   3. a second polynucleotide encoding an alanine racemase which is not native to the host cell, wherein said second polynucleotide is operably linked to a promoter.

In an embodiment of the method of the present invention, the at least one polypeptide of interest is secreted by the bacterial host cell into the fermentation broth.

In an embodiment of the method of the present invention, the method further comprises the step of obtaining the polypeptide of interest from the bacterial host cell culture obtained after step (b), and/or the further step of purifying the polypeptide of interest.

The present invention further relates to a bacterial host cell in which the chromosomal alr gene has been inactivated, wherein said bacterial host cell comprises a plasmid comprising
1. at least one autonomous replication sequence,
2. a first polynucleotide encoding at least one polypeptide of interest, wherein said first polynucleotide is operably linked to a promoter, and
3. a second polynucleotide encoding an alanine racemase which is not native to the host cell, wherein said second polynucleotide is operably linked to a promoter.

In an embodiment of the bacterial host cell of the present invention, the bacterial host cell is obtained or obtainable by carrying out steps a1), a2) and a3) as set forth above.

In one embodiment of the method or the host cell of the present invention, the host cell belongs to the species of Bacillus licheniformis. For example, the host cell is a Bacillus licheniformis strain ATCC14580 (DSM13) host cell. In one embodiment, the host cell belongs to a Bacillus licheniformis species encoding a restriction modification system having a recognition sequence GCNGC.

In an alternative embodiment of the method or the host cell of the present invention, the host cell belongs to the species of Bacillus pumilus.

In one embodiment, the alanine racemase which is not native to the host cell has at least 75% sequence identity to SEQ ID NO: 4.

In one embodiment, the alanine racemase which is not native to the host cell has at least 85% sequence identity to SEQ ID NO: 4.

In one embodiment, the alanine racemase which is not native to the host cell has at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or 100% sequence identity to SEQ ID NO: 4.

In an embodiment of the method or the bacterial host cell of the present invention, the promoter which is operably linked to the polynucleotide encoding the alanine racemase which is not native to the host cell is the promoter of the B. subtilis alrA gene, or a variant thereof having at least 80%, 85%, 90%, 93%, 95%, 98% or 99% sequence identity to said promoter. Preferably, the promoter of the B. subtilis alrA gene comprises a sequence as shown in SEQ ID NO: 46.

In an embodiment of the method or the bacterial host cell of the present invention, the polypeptide of interest is an enzyme. For example, the enzyme may be an enzyme selected from the group consisting of amylase, protease, lipase, mannanase, phytase, xylanase, phosphatase, glucoamylase, nuclease, and cellulase.

In an embodiment of the method or the bacterial host cell of the present invention, the enzyme is protease, such as an aminopeptidase (EC 3.4.11), a dipeptidase (EC 3.4.13), a dipeptidylpeptidase or tripeptidyl-peptidase (EC 3.4.14), a peptidyl-dipeptidase (EC 3.4.15), a serine-type carboxypeptidase (EC 3.4.16), a metallocarboxypeptidase (EC 3.4.17), a cysteine-type carboxypeptidase (EC 3.4.18), an omega peptidase (EC 3.4.19), a serine endopeptidase (EC 3.4.21), a cysteine endopeptidase (EC 3.4.22), an aspartic endopeptidase (EC 3.4.23), a metalloendopeptidase (EC 3.4.24), or a threonine endopeptidase (EC 3.4.25).

The present invention further relates to a fermentation broth comprising the bacterial host cell of the present invention.

### BRIEF DESCRIPTION OF THE DRAWINGS

FIG. 1: Analysis of the protease yield in fed-batch fermentation as described in Example 2 in B. licheniformis in the presence (+) or absence (−) of endogenous alanine racemase genes (alr and ycnD). The protease yield was normalized to the protease yield in B. licheniformis comprising both endogenous genes (BES #158). The protease yield of strain BES #158 was set to 100%.

### DETAILED DESCRIPTION OF THE INVENTION

It is to be understood that as used in the specification and in the claims, “a” or “an” can mean one or more, depending upon the context in which it is used. Thus, for example, reference to “a cell” can mean that at least one cell can be utilized.

Further, it will be understood that the term “at least one” as used herein means that one or more of the items referred to following the term may be used in accordance with the invention. For example, if the term indicates that at least one feed solution shall be used this may be understood as one feed solution or more than one feed solutions, i.e. two, three, four, five or any other number of feed solutions. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, and even more preferably ±5%.

The term “comprising” as used herein shall not be understood in a limiting sense. The term rather indicates that more than the actual items referred to may be present, e.g., if it refers to a method comprising certain steps, the presence of further steps shall not be excluded. However, the term “comprising” also encompasses embodiments where only the items referred to are present, i.e. it has a limiting meaning in the sense of “consisting of”.

As set forth above, the present invention provides for a method for producing at least one polypeptide of interest in a bacterial host cell. The method can be applied for culturing bacterial host cells in both, laboratory and industrial scale fermentation processes. The method comprises the step a) of providing a bacterial host cell as defined above and b) cultivating the bacterial host cell under conditions conducive for maintaining said plasmid in the bacterial host cell and conducive for expressing said at least one polypeptide of interest, thereby producing said at least one polypeptide of interest.

The method according to the present invention may also comprise further steps. Such further steps may encompass the termination of cultivating and/or obtaining the protein of interest from the host cell culture by appropriate purification techniques. Accordingly, the method of the invention may further comprise the step of obtaining the polypeptide of interest from the bacterial host cell culture obtained after step (b). Further, the method may comprise the step of purifying the polypeptide of interest.

The term “alanine racemase” as used herein refers to an enzyme that converts the L-isomer of the amino acid alanine into its D-isomer. Accordingly, an alanine racemase converts L-alanine into D-alanine. An alanine racemase shall have the activity described as EC 5.1.1.1 according to the nomenclature of the International Union of Biochemistry and Molecular Biology (see Recommendations (1992) of the Nomenclature Committee of the International Union of Biochemistry and Molecular Biology including its supplements published 1993-1999)). Whether a polypeptide has alanine racemase activity, or not, can be assessed by well-known alanine racemase assays. In an embodiment, it is assessed as described in the Examples section (see Example 3).

In accordance with the present invention, one chromosomal alr gene encoding for an alanine racemase shall have been inactivated in the bacterial host cell. Host cells belonging to the species Bacillus licheniformiis or Bacillus pumilus naturally comprise two chromosomal genes encoding for two different alanine racemases, Alr and YncD. However, only the chromosomal air gene encoding the Alr alanine racemase shall have been inactivated in the host cell. In other words, the chromosomal yncD gene encoding the YncD alanine racemase shall not have been inactivated in the host cell.

Accordingly, the bacterial host cell provided in step a) of the method of the present invention is obtained or obtainable by the following steps:
a1) providing a Bacillus host cell belonging to the species Bacillus licheniformis or Bacillus pumilus,
a2) inactivating the chromosomal alr gene of said host cell, and
a3) introducing into said host cell a plasmid comprising
   1. at least one autonomous replication sequence,
   2. a first polynucleotide encoding at least one polypeptide of interest, wherein said first polynucleotide is operably linked to a promoter, and
   3. a second polynucleotide encoding an alanine racemase which is not native to the host cell, wherein said second polynucleotide is operably linked to a promoter.

Thus, step a) of the method of the present invention may comprise steps a1), a2) and a3) above.

The term “host cell” in accordance with the present invention refers to a bacterial cell.

In one embodiment, the host cell belongs to the species Bacillus licheniformis, such as a host cell of the Bacillus licheniformis strain as deposited under American Type Culture Collection number ATCC14580 (which is the same as DSM13, see Veith et al. “The complete genome sequence of Bacillus licheniformis DSM13, an organism with great industrial potential.” J. Mol. Microbiol. Biotechnol. (2004) 7:204-211). Alternatively, the host cell may be a host cell of Bacillus licheniformis strain ATCC31972. Alternatively, the host cell may be a host cell of Bacillus licheniformis strain ATCC53757. Alternatively, the host cell may be a host cell of Bacillus licheniformis strain ATCC53926. Alternatively, the host cell may be a host cell of Bacillus licheniformis strain ATCC55768. Alternatively, the host cell may be a host cell of Bacillus licheniformis strain DSM394. Alternatively, the host cell may be a host cell of Bacillus licheniformis strain DSM641. Alternatively, the host cell may be a host cell of Bacillus licheniformis strain DSM1913. Alternatively, the host cell may be a host cell of Bacillus licheniformis strain DSM11259. Alternatively, the host cell may be a host cell of Bacillus licheniformis strain DSM26543.

Preferably, the Bacillus licheniformis strain is selected from the group consisting of Bacillus licheniformis ATCC 14580, ATCC 31972, ATCC 53757, ATCC 53926, ATCC 55768, DSM 13, DSM 394, DSM 641, DSM 1913, DSM 11259, and DSM 26543.

Further, it is envisaged that the host cell as set forth herein belongs to a Bacillus licheniformis species encoding a restriction modification system having a recognition sequence GCNGC.

The coding sequence of the Bacillus licheniformis alr gene is shown in SEQ ID NO: 1. The alanine racemase polypeptide encoded by said gene has an amino acid sequence as shown in SEQ ID NO: 2.

In an alternative embodiment, the host cell belongs to the species Bacillus pumilus (see e.g. Küppers et al., Microb Cell Fact. 2014; 13(1):46, or Schallmey et al., Can J Microbiol. 2004; 50(1):1-17). With respect to Bacillus pumilus, the Alr alanine racemase to be inactivated, preferably, has an amino acid sequence as shown in SEQ ID NO: 47.

The term “inactivating” in connection with the chromosomal alr gene, preferably, means that the enzymatic activity of the Alr alanine racemase encoded by said chromosomal alr gene, respectively, has been reduced as compared to the Alr alanine racemase activity in a control cell. A control cell is a corresponding host cell in which the chromosomal alr gene has not been inactivated, i.e. a corresponding host cell which comprises said chromosomal alr gene. Preferably, the enzymatic activity of the Alr alanine racemase in the bacterial host cell of the present invention has been reduced by at least 40% such as at least 50%, at least 60%, at least 70%, at least 80%, or at least 90% as compared to the corresponding enzymatic activity of Alr in the control cell. More preferably, said enzymatic activity has been reduced by at least 95%. Most preferably, said enzymatic activity has been reduced by 100%, i.e. has been eliminated completely.

The inactivation of the alr gene as referred to herein may be achieved by any method deemed appropriate. In an embodiment, the chromosomal alr gene encoding the Alr alanine racemase has been inactivated by mutation, i.e. by mutating said chromosomal gene. Preferably, said mutation is a deletion, i.e. said chromosomal alr gene has been deleted.

As used herein, the “deletion” of a gene refers to the deletion of the entire coding sequence, deletion of part of the coding sequence, or deletion of the coding sequence including flanking regions. The end result is that the deleted gene is effectively non-functional. In simple terms, a “deletion” is defined as a change in either nucleotide or amino acid sequence in which one or more nucleotides or amino acid residues, respectively, have been removed (i.e., are absent). Thus, a deletion strain has fewer nucleotides or amino acids than the respective wild-type organism.

In another embodiment, the chromosomal alr gene encoding the Alr alanine racemase has been inactivated by gene silencing. Gene silencing can be achieved by introducing into said bacterial host cell antisense expression constructs that result in antisense RNAs complementary to the mRNA of said chromosomal gene, thereby inhibiting expression of said gene. Alternatively, the expression of said gene can be inhibited by blocking transcriptional initiation or transcriptional elongation through the mechanism of CRISPR-inhibition (WO18009520).

The bacterial host cell is typically a wild-type cell comprising the gene deletions in alanine racemase gene. For industrial fermentation processes, the bacterial host cell may be genetically modified to meet the needs of highest product purity and regulatory requirements. It is therefore in scope of the invention to use Bacillus production hosts that may additionally contain modifications, e.g., deletions or disruptions of other genes that may be detrimental to the production, recovery or application of a polypeptide of interest. In one embodiment, a bacterial host cell is a protease-deficient cell. The bacterial host cell, e.g., Bacillus cell, preferably comprises a disruption or deletion of extracellular protease genes including but not limited to aprE, mpr, vpr, bpr, and/or epr. Further preferably the bacterial host cell does not produce spores. Further preferably the bacterial host cell, e.g., a Bacillus cell, comprises a disruption or deletion of spoIIAC, sigE, and/or sigG. Further, preferably the bacterial host cell, e.g., Bacillus cell, comprises a disruption or deletion of one of the genes involved in the biosynthesis of surfactin, e.g., srfA, srfB, srfC, and/or srfD, see, for example, U.S. Pat. No. 5,958,728. It is also preferred that the bacterial host cell comprises a disruption or deletion of one of the genes involved in the biosynthesis of polyglutamic acid. Other genes, including but not limited to the amyEgene, which are detrimental to the production, recovery or application of a polypeptide of interest may also be disrupted or deleted.

The Plasmid

The bacterial host cell as referred to herein shall comprise a plasmid. Said plasmid shall comprise i) at least one autonomous replication sequence, ii) a polynucleotide encoding at least one polypeptide of interest, operably linked to a promoter, and iii) a polynucleotide encoding an alanine racemase which is not native to the host cell, operably linked to a promoter.

As used herein, the term “plasmid” refers to an extrachromosomal circular DNA that is autonomously replicating in the host cell. Thus, a plasmid is understood as extrachromosomal vector (and shall not be stably integrated in the bacterial chromosome).

In accordance with the present invention, the replication of a plasmid shall be independent of the replication of the chromosome of the bacterial host cell. For autonomous replication, the plasmid comprises an autonomous replication sequence, i.e. an origin of replication enabling the plasmid to replicate autonomously in the bacterial host cell. Examples of bacterial origins of replication are the origins of replication of plasmids pUB110, pBC16, pE194, pC194, pTB19, pAMR1, pTA1060 permitting replication in Bacillus and plasmids pBR322, colE1, pUC19, pSC101, pACYC177, and pACYC184 permitting replication in E. ° coli (see e.g. Sambrook, J. and Russell, D. W. Molecular cloning. A laboratory manual, 3rd ed, Cold Spring Harbor Laboratory Press, Cold Spring Harbor, N.Y. 2001.). The copy number of a plasmid is defined as the average number of plasmids per bacterial cell or per chromosome under normal growth conditions. Moreover, there are different types of replication origins that result in different copy numbers in the bacterial host. The plasmid replicon pBS72 (accession number AY102630.1) and the plasmids pTB19 and derivatives pTB51, pTB52 confer low copy number with 6 copies and 1 to 8 copies, respectively, within Bacillus cells whereas plasmids pE194 (accession number V01278.1) and pUB110 (accession number M19465.1)/pBC16 (accession number U32369.1) confer low-medium copy number with 14-20 and medium copy number with 30-50 copies per cell, respectively. Plasmid pE194 was analyzed in more detail (Villafane, et al (1987): J. Bacteriol. 169(10), 4822-4829) and several pE194—cop mutants described having high copy numbers within Bacillus ranging from 85 copies to 202 copies. Moreover, plasmid pE194 is temperature sensitive with stable copy number up to 37° C., however, abolished replication above 43° C. In addition, it exists a pE194 variant referred to as pE194ts with two point mutations within the cop-repF region (nt 1235 ad nt 1431) leading to a more drastic temperature sensitivity—stable copy number up to 32° C., however, only 1 to 2 copies per cell at 37° C.

In some embodiments, the autonomous replication sequence comprised by the plasmid confers a low copy number in the bacterial host cell, such as 1 to 8 copies of the plasmid in the bacterial host cell.

In some embodiments, the autonomous replication sequence confers a low medium copy number in the bacterial cell, such as 9 to 20 copies of the plasmid in the bacterial host cell.

In some embodiments, the autonomous replication sequence confers a medium copy number in the bacterial cell, such as 21 to 60 copies of the plasmid in the bacterial host cell.

In some embodiments, the autonomous replication sequence confers a high copy number in the bacterial cell, such as 61 or more copies of the plasmid in the bacterial host cell.

In a preferred embodiment, the plasmid comprises replicon pBS72 (accession number AY102630.1) as autonomous replication sequence. In another preferred embodiment, the plasmid comprises the replication origin of pUB110 (accession number M19465.1)/pBC16 (accession number U32369.1) as autonomous replication sequence.

The plasmid can be introduced into the host cell by any method suitable for transferring the plasmid into the cell, for example, by transformation using electroporation, protoplast transformation or conjugation.

The Polypeptide of Interest

In addition to the at least one autonomous replication sequence, the plasmid as referred to herein shall comprise at least one polynucleotide encoding a polypeptide of interest (operably linked to a promoter).

The terms “polynucleotide”, “nucleic acid sequence”, “nucleotide sequence”, “nucleic acid”, “nucleic acid molecule” are used interchangeably herein and refer to nucleotides, typically deoxyribonucleotides, in a polymeric unbranched form of any length. The terms “polypeptide” and “protein” are used interchangeably herein and refer to amino acids in a polymeric form of any length, linked together by peptide bonds.

The terms “coding for” and “encoding” are used interchangeably herein. Typically, the terms refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein, if transcription and translation of mRNA corresponding to that gene produces the protein in a cell or other biological system.

The term “polypeptide of interest” as used herein refers to any protein, peptide or fragment thereof which is intended to be produced in the bacterial host cell. A protein, thus, encompasses polypeptides, peptides, fragments thereof as well as fusion proteins and the like.

Preferably, the polypeptide of interest is an enzyme. In a particular embodiment, the enzyme is classified as an oxidoreductase (EC 1), a transferase (EC 2), a hydrolase (EC 3), a lyase (EC 4), an isomerase (EC 5), or a ligase (EC 6). In a preferred embodiment, the protein of interest is an enzyme suitable to be used in detergents.

Most preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, glucoamylase, nuclease, and cellulase, preferably, amylase or protease, preferably, a protease. Most preferred is a serine protease (EC 3.4.21), preferably a subtilisin protease.

In particular, the following proteins of interest are preferred:

Enzymes having proteolytic activity are called “proteases” or “peptidases”. Proteases are active proteins exerting “protease activity” or “proteolytic activity”. Proteases are members of class EC 3.4. Proteases include aminopeptidases (EC 3.4.11), dipeptidases (EC 3.4.13), dipeptidylpeptidases and tripeptidyl-peptidases (EC 3.4.14), peptidyl-dipeptidases (EC 3.4.15), serinetype carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metalloendopeptidases (EC 3.4.24), threonine endopeptidases (EC 3.4.25), endo-peptidases of unknown catalytic mechanism (EC 3.4.99). Commercially available protease enzymes include but are not limited to Lavergy™ Pro (BASF); Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coro-nase®, Coronase® Ultra, Neutrase®, Everlase® and Esperase® (Novozymes A/S), those sold under the tradename Maxatase®, Maxacal®, Maxapem®, Purafect®, Purafect® Prime, Pura-fect MA®, Purafect Ox®, Purafect OxP®, Puramax®, Properase®, FN2®, FN3®, FN4®, Ex-cellase®, Eraser®, Ultimase®, Opticlean®, Effectenz®, Preferenz® and Optimase® (Dan-isco/DuPont), Axapem™ (Gist-Brocases N. V.), Baccillus lentus Alkaline Protease, and KAP (Bacillus alkalophilus subtilisin) from Kao. At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5,) and subtilisin (also known as subtilopeptidase, e.g., EC 3.4.21.62), the latter hereinafter also being referred to as “subtilisin”. Proteases according to the invention have proteolytic activity. The methods for determining proteolytic activity are well-known in the literature (see e.g. Gupta et al. (2002), Appl. Microbiol. Bio-technol. 60: 381-395).

In an embodiment, the polynucleotide encoding at least one polypeptide of interest is heterologous to the bacterial host cell. The term “heterologous” (or exogenous or foreign or recombinant or non-native) polypeptide or protein as used throughout the specification is defined herein as a polypeptide or protein that is not native to the host cell. Similarly, the term “heterologous” (or exogenous or foreign or recombinant or non-native) polynucleotide refers to a polynucleotide that is not native to the host cell.

In another embodiment, the polynucleotide encoding the polypeptide of interest is native to the bacterial host cell. Thus, the polynucleotide encoding the polypeptide of interest may be native to the host cell. The term “native” (or wildtype or endogenous) polynucleotide or polypeptide as used throughout the specification refers to the polynucleotide or polypeptide in question as found naturally in the host cell. However, since the polynucleotide has been introduced into the host cell on a plasmid, the “native” polynucleotide or polypeptide is still considered as recombinant.

The Alanine Racemase which is not Native to the Host Cell

In addition to the at least one autonomous replication sequence and the at least one polynucleotide encoding a polypeptide of interest, the plasmid as referred to herein shall comprise a polynucleotide encoding an alanine racemase which is not native to the host cell. Said polynucleotide shall be operably linked to a suitable promoter, such as a constitutive promoter.

The term “alanine racemase” has been defined above. In an embodiment, the alanine racemase which is not native to the host cell is heterologous with respect to the bacterial host cell. Accordingly, the amino acid sequence of the alanine racemase which is not native to the host cell differs from the amino acid sequence of the Alr alanine racemase. Further, the amino acid sequence shall differ from the sequence of the Alr alanine racemase. For example, the alanine racemase which is not native to the host cell shows less than 90% sequence identity to the Alr (and YncD) alanine racemase.

In some embodiments, the alanine racemase which is not native to the host cell is a bacterial alanine racemase. A suitable bacterial alanine racemase can be, for example, identified by carrying out the in silico analysis described in Example 4. Accordingly, it may show a significant alignment against COG0787 (see Example for more details).

The alanine racemase which is not native to the host cell may be any alanine racemase as long as it has alanine racemase activity. In a preferred embodiment, the alanine racemase which is not native to the host cell is a bacterial alanine racemase. Preferred amino acid sequences are shown in Table 3 and, in particular, in Table 4.

In an embodiment, the alanine racemase which is not native to the host cell comprises an amino acid sequence as shown in SEQ ID NO: 4, 47, 48, 49, 50, 51, 52 or 53, or is a variant thereof. In particular, the alanine racemase which is not native to the host cell comprises an amino acid sequence as shown in SEQ ID NO: 4, or is a variant thereof.

The alanine racemases having an amino acid sequence as shown in SEQ ID NO: 4, 47, 48, 49, 50, 51, 52 or 53 are herein also referred to as “parent enzymes” or “parent sequences. “Parent” sequence (e.g., “parent enzyme” or “parent protein”) is the starting sequence for introduction of changes (e.g. by introducing one or more amino acid substitutions) of the sequence resulting in “variants” of the parent sequences. Thus, the term “enzyme variant” or “sequence variant” or “protein variant” are used in reference to parent enzymes that are the origin for the respective variant enzymes. Therefore, parent enzymes include wild type enzymes and variants of wildtype enzymes which are used for development of further variants. Variant enzymes differ from parent enzymes in their amino acid sequence to a certain extent; however, variants at least maintain the enzyme properties of the respective parent enzyme. In one embodiment, enzyme properties are improved in variant enzymes when compared to the respective parent enzyme. In one embodiment, variant enzymes have at least the same enzymatic activity when compared to the respective parent enzyme or variant enzymes have increased enzymatic activity when compared to the respective parent enzyme.

Variants of a parent enzyme molecule (e.g. the alanine racemase which is not native to the host cell having amino acid sequence as shown in SEQ ID NO: 4, 47, 48, 49, 50, 51, 52 or 53) may have an amino acid sequence which is at least n percent identical to the amino acid sequence of the respective parent enzyme having enzymatic activity with n being an integer between 50 and 100, preferably 50, 55, 60, 65, 70, 75, 80, 85, 90, 91, 92, 93, 94, 95, 96, 97, 98 or 99 compared to the full length polypeptide sequence. Variant enzymes described herein which are n percent identical when compared to a parent enzyme have enzymatic activity.

In some embodiments, a variant of the alanine racemase which is not native to the host cell comprises an amino acid sequence which is at least 70%, 75% 80%, 85%, 90%, 95% or 98% identical to an amino acid sequence as shown in SEQ ID NO: 4, 47, 48, 49, 50, 51, 52 or 53 (preferably, to SEQ ID NO: 4).

Enzyme variants may be, thus, defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSU M62). The preferred alignment for the purpose of this invention is that alignment, from which the highest sequence identity can be determined.

After aligning the two sequences, in a second step, an identity value shall be determined from the alignment. Therefore, according to the present invention the following calculation of percentidentity applies:

%-identity=(identical residues/length of the alignment region which is showing the respective sequence of this invention over its complete length)*100. Thus, sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

For calculating the percent identity of two DNA sequences the same applies as for the calculation of percent identity of two amino acid sequences with some specifications. For DNA sequences encoding for a protein the pairwise alignment shall be made over the complete length of the coding region from start to stop codon excluding introns. For non-protein-coding DNA sequences, the pairwise alignment shall be made over the complete length of the sequence of this invention, so the complete sequence of this invention is compared to another sequence, or regions out of another sequence. Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

Enzyme variants may be defined by their sequence similarity when compared to a parent enzyme. Sequence similarity usually is provided as “% sequence similarity” or “%-similarity”. For calculating sequence similarity in a first step, a sequence alignment has to be generated as described above. In a second step, the percent-similarity has to be calculated, whereas percent sequence similarity takes into account that defined sets of amino acids share similar properties, e.g., by their size, by their hydrophobicity, by their charge, or by other characteristics. Herein, the exchange of one amino acid with a similar amino acid is referred to as “conservative mutation”. Enzyme variants comprising conservative mutations appear to have a minimal effect on protein folding resulting in certain enzyme properties being substantially maintained when compared to the enzyme properties of the parent enzyme.

For determination of %-similarity according to this invention the following applies, which is also in accordance with the BLOSUM62 matrix, which is one of the most used amino acids similarity matrix for database searching and sequence alignments
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W.

Conservative amino acid substitutions may occur over the full length of the sequence of a polypeptide sequence of a functional protein such as an enzyme. In one embodiment, such mutations are not pertaining to the functional domains of an enzyme. In another embodiment, conservative mutations are not pertaining to the catalytic centers of an enzyme.

Therefore, according to the present invention the following calculation of percent-similarity applies:
%-similarity=[(identical residues+similar residues)/length of the alignment region which is showing the respective sequence of this invention over its complete length]*100. Thus, sequence similarity in relation to comparison of two amino acid sequences herein is calculated by dividing the number of identical residues plus the number of similar residues by the length of the alignment region which is showing the respective sequence of this invention over its complete length. This value is multiplied with 100 to give “%-similarity”.

Especially, variant enzymes comprising conservative mutations which are at least m percent similar to the respective parent sequences with m being an integer between 70 and 100, preferably 70, 75, 80, 85, 90, 91, 92, 93, 94, 95, 96, 97, 98 or 99 compared to the full length polypeptide sequence, are expected to have essentially unchanged enzyme properties. Variant enzymes described herein with m percent-similarity when compared to a parent enzyme, have enzymatic activity.

The Promoter

The polynucleotide encoding the polypeptide of interest and the polynucleotide encoding the alanine racemase which is not native to the host cell shall be expressed in the bacterial host cell. Accordingly, both the polynucleotide encoding the polypeptide of interest and the polynucleotide encoding the alanine racemase which is not native to the host cell shall be operably linked to a promoter.

The term “operably linked” as used herein refers to a functional linkage between the promoter sequence and the gene of interest, such that the promoter sequence is able to initiate transcription of the gene of interest.

A “promoter” or “promoter sequence” is a nucleotide sequence located upstream of a gene on the same strand as the gene that enables that gene's transcription. Promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

A promoter can be an “inducer-dependent promoter” or an “inducer-independent promoter” comprising constitutive promoters or promoters which are under the control of other cellular regulating factors. In an embodiment, the promoter is a constitutive promoter.

The person skilled in the art is capable to select suitable promoters for expressing the alanine racemase which is not native to the host cell and the polypeptide of interest. For example, the polynucleotide encoding the polypeptide of interest is, preferably, operably linked to an “inducer-dependent promoter” or an “inducer-independent promoter”. Further, the polynucleotide encoding the alanine racemase which is not native to the host cell is, preferably, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule which presence in the fermentation medium is capable of affecting an increase in expression of a gene by increasing the activity of an inducer-dependent promoter operably linked to the gene. Preferably, the inducer molecule is a carbohydrate or an analog thereof. In one embodiment, the inducer molecule is a secondary carbon source of the Bacillus cell. In the presence of a mixture of carbohydrates, cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Examples of inducer dependent promoters are given in the table below by reference to the respective operon:

| Operon | Regulator a) | Type b) | Inducer | Organism |
|---|---|---|---|---|
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β-glucosides | B. subtilis |
| levDEFG sacL | LevR | A | fructose | B. subtilis |
| mtlAD | MtlR | A | mannitol | B. subtilis |
| manPA-yjdF | ManR | A | mannose | B. subtilis |
| manR | ManR | A | mannose | B. subtilis |
| bglFB bgIG | BgIG | AT | β-glucosides | E. coli |
| lacTEGF | LacT | AT | lactose | L. casei |
| lacZYA | lacI | R | Allolactose; IPTG | E. coli |
| | | | (Isopropyl β-D-1- | |
| | | | thiogalacto- | |
| | | | pyranoside) | |
| araBAD | araC | AR | L-arabinose | E. coli |
| xylAB | XyIR | R | Xylose | B. subtilis |

a: transcriptional regulator protein
b: A: activator
AT: antiterminator
R: repressor
AR: activator/repressor

In contrast thereto, the activity of promoters that do not depend on the presence of an inducer molecule (herein called ‘inducer-independent promoters’) are either constitutively active or can be increased regardless of the presence of an inducer molecule that is added to the fermentation medium.

Constitutive promoters, typically, are independent of other cellular regulating factors and transcription initiation is dependent on sigma factor A (sigA). The sigA-dependent promoters comprise the sigma factor A specific recognition sites ‘-35’-region and ‘-10’-region.

Preferably, the ‘inducer-independent promoter’ sequence is selected from the group consisting of constitutive promoters not limited to promoters Pveg, PlepA, PserA, PymdA, Pfba and derivatives thereof with different strength of gene expression (Guiziou et al, (2016): Nucleic Acids Res. 44(15), 7495-7508), the aprEpromoter, the bacteriophage SPO1 promoters P4, P5, P15 (WO15118126), the cryll IA promoter from Bacillus thuringiensis (WO9425612), the amyQ promoter from Bacillus amyloliquefaciens, the amyL promoter and promoter variants from Bacillus licheniformis (U.S. Pat. No. 5,698,415) and combinations thereof, or active fragments or variants thereof, preferably an aprE promoter sequence.

In a preferred embodiment, the inducer-independent promoter is an aprE promoter.

An “aprE promoter” or “aprE promoter sequence” is the nucleotide sequence (or parts or variants thereof) located upstream of an aprEgene, i.e., a gene coding for a Bacillus subtilisin Carlsberg protease, on the same strand as the aprE gene that enables that aprEgene's transcription.

The native promoter from the gene encoding the Carlsberg protease, also referred to as aprE promoter, is well described in the art. The aprEgene is transcribed by sigma factor A (sigA) and its expression is highly controlled by several regulators—DegU acting as activator of aprE expression, whereas AbrB, ScoC (hpr) and SinR are repressors of aprEexpression.

WO9102792 discloses the functionality of the promoter of the alkaline protease gene for the large-scale production of subtilisin Carlsberg-type protease in Bacillus licheniformis. In particular, WO9102792 describes the 5′ region of the subtilisin Carlsberg protease encoding aprE gene of Bacillus licheniformis (FIG. 27) comprising the functional aprEgene promoter and the 5′UTR comprising the ribosome binding site (Shine Dalgarno sequence).

Further, the promoter to be used may be the endogenous promoter from the polynucleotide to be expressed. As set forth above, the alanine racemase which is not native to the host cell may be a bacterial alanine racemase. Thus, the polynucleotide encoding said bacterial alanine racemase may be operably linked to the endogenous, i.e. native, promoter of the gene encoding the bacterial alanine racemase.

In a preferred embodiment, the polynucleotide encoding the alanine racemase which is not native to the host cell is operably linked to an alr promoter, such as a Bacillus alr promoter. For example, the promoter is the Bacillus subtilis alrA promoter, or a variant thereof. Preferably, the alrA promoter from Bacillus subtilis comprises a nucleic acid sequence as shown in SEQ ID NO: 46. A variant of this promoter, preferably, comprises a nucleic acid sequence having at least 80%, 85%, 90%, 93%, 95%, 98% or 99% sequence identity to nucleic acid sequence as shown in SEQ ID NO: 46.

In case, the polynucleotide encoding the alanine racemase which is not native to the host cell is the Bacillus subtilis alrA gene, the promoter may be thus the native promoter.

The term “transcription start site” or “transcriptional start site” shall be understood as the location where the transcription starts at the 5′ end of a gene sequence. In prokaryotes the first nucleotide, referred to as +1 is in general an adenosine (A) or guanosine (G) nucleotide. In this context, the terms “sites” and “signal” can be used interchangeably herein.

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

Further, optionally, the promoter comprises a 5′UTR. This is a transcribed but not translated region downstream of the −1 promoter position. Such untranslated region for example should contain a ribosome binding site to facilitate translation in those cases where the target gene codes for a peptide or polypeptide.

With respect to the 5′UTR the invention in particular teaches to combine the promoter of the present invention with a 5′UTR comprising one or more stabilizing elements. This way the mRNAs synthesized from the promoter region may be processed to generate mRNA transcript with a stabilizer sequence at the 5′ end of the transcript. Preferably such a stabilizer sequence at the 5′end of the mRNA transcripts increases their half-life as described by Hue et al, 1995, Journal of Bacteriology 177: 3465-3471. Suitable mRNA stabilizing elements are those described in
WO08148575, preferably SEQ ID NO. 1 to 5 of WO08140615, or fragments of these sequences which maintain the mRNA stabilizing function, and in
WO08140615, preferably Bacillus thuringiensis CryIIIA mRNA stabilising sequence or bacteriophage SP82 mRNA stabilising sequence, more preferably a mRNA stabilizing sequence according to SEQ ID NO. 4 or 5 of WO08140615, more preferably a modified mRNA stabilizing sequence according to SEQ ID NO. 6 of WO08140615, or fragments of these sequences which maintain the mRNA stabilizing function.

Preferred mRNA stabilizing elements are selected from the group consisting of aprE, grpE, cotG, SP82, RSBgsiB, CryIIIA mRNA stabilizing elements, or according to fragments of these sequences which maintain the mRNA stabilizing function. A preferred mRNA stabilizing element is the grpE mRNA stabilizing element (corresponding to SEQ ID NO. 2 of WO08148575).

The 5′UTR also preferably comprises a modified rib leader sequence located downstream of the promoter and upstream of an ribosome binding site (RBS). In the context of the present invention, a rib leader is herewith defined as the leader sequence upstream of the riboflavin biosynthetic genes (rib operon) in a Bacillus cell, more preferably in a Bacillus subtilis cell. In Bacillus subtilis, the rib operon, comprising the genes involved in riboflavin biosynthesis, include ribG (ribD), ribB (ribE), ribA, and ribH genes. Transcription of the riboflavin operon from the rib promoter (Prib) in B. subtilis is controlled by a riboswitch involving an untranslated regulatory leader region (the rib leader) of almost 300 nucleotides located in the 5′-region of the rib operon between the transcription start and the translation start codon of the first gene in the operon, ribG. Suitable rib leader sequences are described in WO2015/1181296, in particular pages 23-25, incorporated herein by reference.

In step b) of the method of the present invention, the bacterial host cell is cultivated under conditions which are conducive for maintaining said plasmid in the bacterial host cell and for expressing said at least one polypeptide of interest. Thereby, the at least one polypeptide of interest is produced.

The term “cultivating” as used herein refers to keeping alive and/or propagating the bacterial host cell comprised in a culture at least for a predetermined time. The term encompasses phases of exponential cell growth at the beginning of growth after inoculation as well as phases of stationary growth. The person skilled in the art is capable of selecting conditions which allow for maintaining said plasmid in the bacterial host cell and for expressing said at least one polypeptide of interest. Preferably, the conditions are selective for maintaining said plasmid in said host cell. The conditions may depend on the bacterial host cell strain. An exemplary cultivation medium and exemplary cultivation conditions for Bacillus licheniformis are disclosed in Example 2. In order to allow for maintaining the plasmid in the bacterial host cell, the bacterial host cell is preferably cultivated in the absence of extraneously added D-alanine, i.e. no D-alanine has been added to the cultivation medium.

Further, it is envisaged that the cultivation is carried out in the absence of antibiotics. Thus, it is envisaged that the plasmid as referred to herein does not comprise antibiotic resistance genes.

The method of the present invention, if applied, allows for increasing the expression, i.e. the production, of the at least one polypeptide of interest. Preferably, expression is increased as compared to a control cell. A control cell may be a control cell of the same species in which the two chromosomal alanine racemase genes have not been inactivated. In a preferred embodiment, expression of the at least one polypeptide of interest is increased by at least 4%, such as by at least 6%, such as by at least 7% as compared to the expression in the control cell. For example, expression of the at least one polypeptide of interest may be increased by 4% to 10%, such as by 6% to 10%, as compared to the control cell. The expression can be measured by determining the amount of the polypeptide in the host cell and/or in the cultivation medium.

The definitions and explanations given herein above, preferably, apply mutatis mutandis to the following:

The present invention further relates to a Bacillus host cell belonging to the species Bacillus licheniformis or Bacillus pumilus, in which the chromosomal alr gene has been inactivated and which preferably comprises a plasmid comprising
1. at least one autonomous replication sequence,
2. a first polynucleotide encoding at least one polypeptide of interest, wherein said first polynucleotide is operably linked to a promoter, and
3. a second polynucleotide encoding an alanine racemase which is not native to the host cell, wherein said second polynucleotide is operably linked to a promoter.

The host cell is preferably obtained or obtainable by carrying out the following steps:
a1) providing a Bacillus host cell belonging to the species Bacillus licheniformis or Bacillus pumilus,
a2) inactivating the chromosomal alr gene of said host cell, and
a3) introducing into said host cell said plasmid.

Preferably, the bacterial host cell expresses the at least one polypeptide of interest and the alanine racemase which is not native to the host cell. More preferably, the expression of the at least one polypeptide of interest is increased as compared to the expression in a control cell (as described elsewhere herein).

Alternatively, the host cell may be used for locus expansion (as described e.g. in WO09120929). Accordingly, the host cell may comprise
u) a non-replicative vector comprising
u1) optionally, a plus origin of replication (ori+),
u2) a polynucleotide encoding at least one polypeptide of interest, operably linked to a promoter,
u3) a polynucleotide encoding an alanine racemase which is not native to the host cell, operably linked to a promoter, and
u4) a polynucleotide which has homology to a chromosomal polynucleotide of the bacterial host cell to allow integration of the non-replicative vector into the chromosome of the bacterial host cell by recombination.

### EXAMPLES

Materials and Methods

Unless otherwise stated, the following experiments have been performed by applying standard equipment, methods, chemicals, and biochemicals as used in genetic engineering and fermentative production of chemical compounds by cultivation of microorganisms. See also Sambrook et al. (Sambrook, J. and Russell, D. W. Molecular cloning. A laboratory manual, 3^{rd} ed, Cold Spring Harbor Laboratory Press, Cold Spring Harbor, N.Y. 2001).

Electrocompetent Bacillus licheniformis cells and electroporation Transformation of DNA into B. licheniformis ATCC53926 is performed via electroporation. Preparation of electrocompetent B. licheniformis ATCC53926 cells and transformation of DNA is performed as essentially described by Brigidi et al. (Brigidi, P., Mateuzzi, D. (1991), Biotechnol. Techniques 5, 5) with the following modification: Upon transformation of DNA, cells are recovered in 1 ml LBSPG buffer and incubated for 60 min at 37° C. (Vehmaanpera J., 1989, FEMS Microbio. Lett., 61: 165-170) following plating on selective LB-agar plates. B. licheniformis strains defective in alanine racemase, 100 μg/ml D-alanine was added to all cultivation media, cultivation-agar plates and buffers. Upon transformation of plasmids carrying the alanine racemase gene, e.g. pUA58P, D-alanine was added in recovery LBSPG buffer, however, not on selection plates.

In order to overcome the Bacillus licheniformis specific restriction modification system of Bacillus licheniformis strain ATCC53926, plasmid DNA is isolated from Ec #098 cells or B. subtilis Bs #056 cells as described below.

Plasmid Isolation

Plasmid DNA was isolated from Bacillus and E. coli cells by standard molecular biology methods described in (Sambrook, J. and Russell, D. W. Molecular cloning. A laboratory manual, 3^{rd} ed, Cold Spring Harbor Laboratory Press, Cold Spring Harbor, N.Y. 2001) or the alkaline lysis method (Birnboim, H. C., Doly, J. (1979), Nucleic Acids Res 7(6): 1513-1523). Bacillus cells were in comparison to E. coli treated with 10 mg/ml lysozyme for 30 min at 37° C. prior to cell lysis.

Molecular Biology Methods and Techniques

Standard methods in molecular biology not limited to cultivation of Bacillus and E. coli microorganisms, electroporation of DNA, isolation of genomic and plasmid DNA, PCR reactions, and cloning technologies were performed as essentially described by Sambrook and Rusell (see above).

Strains

B. subtilis Strain Bs #056

The prototrophic Bacillus subtilis strain KO-7S (BGSCID: 1S145; Zeigler D. R.) was made competent according to the method of Spizizen (Anagnostopoulos, C. and Spizizen, J. (1961). J. Bacteriol. 81, 741-746.) and transformed with the linearized DNA-methyltransferase expression plasmid pMIS012 for integration of the DNA-methyltransferase into the amyEgene as described for the generation of B. subtils Bs #053 in WO2019/016051. Cells were spread and incubated overnight at 37° C. on LB-agar plates containing 10 μg/ml chloramphenicol. Grown colonies were picked and stroke on both LB-agar plates containing 10 μg/ml chloramphenicol and LB-agar plates containing 10 μg/ml chloramphenicol and 0.5% soluble starch (Sigma) following incubation overnight at 37° C. The starch plates were covered with iodine containing Lugols solution and positive integration clones identified with negative amylase activity. Genomic DNA of positive clones was isolated by standard phenol/chloroform extraction methods after 30 min treatment with lysozyme (10 mg/ml) at 3° C., following analysis of correct integration of the MTase expression cassette by PCR. The resulting B. subtilis strain is named Bs #056.

E. coli Strain Ec #098

E. coli strain Ec #098 is an E. coli INV110 strain (Invitrogen/Life technologies) carrying the DNA-methyltransferase encoding expression plasmid pMDS003 WO2019016051.

Generation of B. licheniformis Gene Knock-Out Strains

For gene deletion in B. licheniformis strain ATCC53926 (U.S. Pat. No. 5,352,604) and derivatives thereof, deletion plasmids were transformed into E. coli strain Ec #098 made competent according to the method of Chung (Chung, C. T., Niemela, S. L., and Miller, R. H. (1989). One-step preparation of competent Escherichia coli: transformation and storage of bacterial cells in the same solution. PNAS 86, 2172-2175), following selection on LB-agar plates containing 100 μg/ml ampicillin and 30 μg/ml chloramphenicol at 37° C. Plasmid DNA was isolated from individual clones and analyzed for correctness by PCR analysis. The isolated plasmid DNA carries the DNA methylation pattern of B. licheniformis ATCC53926 and is protected from degradation upon transfer into B. licheniformis.

aprE Gene Deletion Strain Bk #002

Electrocompetent B. licheniformis ATCC53926 cells (U.S. Pat. No. 5,352,604) were prepared as described above and transformed with 1 μg of pDel003 aprEgene deletion plasmid isolated from E. coli Ec #098 following plating on LB-agar plates containing 5 μg/ml erythromycin at 37° C. The gene deletion procedure was performed as described in the following: Plasmid carrying B. licheniformis cells were grown on LB-agar plates with 5 μg/ml erythromycin at 45° C. forcing integration of the deletion plasmid via Campbell recombination into the chromosome with one of the homology regions of pDel003 homologous to the sequences 5′ or 3′ of the aprEgene. Clones were picked and cultivated in LB-media without selection pressure at 45° C. for 6 hours, following plating on LB-agar plates with 5 μg/ml erythromycin at 30° C. Individual clones were picked and analyzed by colony-PCR with oligonucleotides SEQ ID NO: 27 and SEQ ID NO: 28 for successful deletion of the aprEgene. Putative deletion positive individual clones were picked and taken through two consecutive overnight incubation in LB media without antibiotics at 45° C. to cure the plasmid and plated on LB-agar plates for overnight incubation at 30° C. Single clones were again restreaked on LB-agar plates with 5 μg/ml erythromycin and analyzed by colony PCR for successful deletion of the aprEgene. A single erythromycin-sensitive clone with the correct deleted aprEgene was isolated and designated Bli #002.

amyB Gene Deletion Strain Bli #003

Electrocompetent B. licheniformis Bli #002 cells were prepared as described above and transformed with 1 μg of pDel004 amyB gene deletion plasmid isolated from E. coli Ec #098 following plating on LB-agar plates containing 5 μg/ml erythromycin at 30° C. The gene deletion procedure was performed as described for the aprEgene. The deletion of the amyB gene was analyzed by PCR with oligonucleotides SEQ ID NO: 30 and SEQ ID NO: 31. The resulting B. licheniformis strain with a deleted aprE and deleted amyB gene is designated Bli #003.

sigF Gene Deletion Strain Bli #004

Electrocompetent B. licheniformis Bli #003 cells were prepared as described above and transformed with 1 μg of pDel005 sigF gene deletion plasmid isolated from E. coli Ec #098 following plating on LB-agar plates containing 5 μg/ml erythromycin at 30° C. The gene deletion procedure was performed as described for the aprEgene. The deletion of the sigF gene was analyzed by PCR with oligonucleotides SEQ ID NO: 33 and SEQ ID NO: 34. The resulting B. licheniformis strain with a deleted aprE, a deleted amyB gene and a deleted sigF gene is designated Bli #004. B. licheniformis strain Bli #004 is no longer able to sporulate as described (Fleming, A. B., M. Tangney, P. L. Jorgensen, B. Diderichsen, and F. G. Priest. 1995. Extracellular enzyme synthesis in a sporulation-deficient strain of Bacillus licheniformis. Appl. Environ. Microbiol. 61: 3775-3780).

Poly-Gamma Glutamate Synthesis Genes Deletion Strain Bli #008

Electrocompetent Bacillus licheniformis Bli #004 cells were prepared as described above and transformed with 1 μg of pDel007 pga gene deletion plasmid isolated from E. coli Ec #098 following plating on LB-agar plates containing 5 μg/ml erythromycin at 30° C.

The gene deletion procedure was performed as described for the deletion of the aprEgene. The deletion of the pga genes was analyzed by PCR with oligonucleotides SEQ ID NO: 36 and SEQ ID NO: 37. The resulting Bacillus licheniformis strain with a deleted aprE, a deleted amyB gene, a deleted sigF gene and a deleted pga gene cluster is designated Bli #008.

air gene deletion strain Bli #071

Electrocompetent B. licheniformis Bli #008 cells were prepared as described above and transformed with 1 μg of pDel0035 alr gene deletion plasmid isolated from E. coli Ec #098 following plating on LB-agar plates containing 5 μg/ml erythromycin at 30° C. The gene deletion procedure was performed as described for the aprEgene, however, all media and media-agar plates were in addition supplemented with 100 μg/ml D-alanine (Ferrari, 1985). The deletion of the alr gene was analyzed by PCR with oligonucleotides SEQ ID NO: 39 and SEQ ID NO: 40. The resulting B. licheniformis strain with a deleted aprE, a deleted amyB gene, a deleted sigF gene, a deleted pga gene cluster and a deleted alr gene is designated B. licheniformis Bli #071.

yncD Gene Deletion Strain Bli #073

Electrocompetent B. licheniformis Bli #008 cells were prepared as described above, however, at all times media, buffers and solution were supplemented with 100 μg/ml D-alanine. Electrocompetent Bli #008 cells were transformed with 1 μg of pDel0036 yncD gene deletion plasmid isolated from E. coli Ec #098 following plating on LB-agar plates containing 5 μg/ml erythromycin and 100 μg/ml D-alanine at 30° C.

The gene deletion procedure was performed as described for the aprEgene, however, all media and media-agar plates were in addition supplemented with 100 μg/ml D-alanine. The deletion of the yncD gene was analyzed by PCR with oligonucleotides SEQ ID NO: 42 and SEQ ID NO: 43. The resulting B. licheniformis strain with a deleted aprE, a deleted amyB gene, a deleted sigF gene, a deleted pga gen cluster and a deleted yncD is designated B. licheniformis Bli #073.

Plasmids

Plasmid pUK57S: Type-II-Assembly Destination Shuttle Plasmid

The BsaI site within the repU gene as well as the BpiI site 5′ of the kanamycin resistance gene of the protease expression plasmid pUK56S (WO2019016051) were removed in two sequential rounds by applying the Quickchange mutagenesis Kit (Agilent) with quickchange oligonucleotides SEQ ID NO: 10, SEQ ID NO: 11, SEQ ID NO: 12, SEQ ID NO: 13, respectively. Subsequently, the plasmid was restricted with restriction endonuclease NdeI and SacI following ligation with a modified type-II assembly mRFP cassette, cut with enzymes NdeI and SacI.

The modified mRFP cassette (SEQ ID NO: 14) comprises the mRPF cassette from plasmid pBSd141R (Accession number: KY995200, Radeck, J., D. Meyer, N. Lautenschlager, and T. Mascher. 2017. Bacillus SEVA siblings: A Golden Gate-based toolbox to create personalized integrative plasmids for Bacillus subtilis. Sci. Rep. 7: 14134) with flanking type-11 restriction enzyme sites of BpiI, the terminator region of the aprE gene from Bacillus licheniformis and flanking NdeI and SacI sites and was ordered as gene synthesis fragment (Geneart, Regensburg). The ligation mixture was transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid is named pUK57S.

Plasmid pUK57: Type-II-Assembly Destination Bacillus Plasmid

The backbone of pUK57S was PCR-amplified with oligonucleotides SEQ ID NO: 15 and SEQ ID NO: 16 comprising additional EcoRI sites. After EcoRI and DpnI restriction, the PCR fragment was ligated using T4 ligase (NEB) following transformation into B. subtilis Bs #056 cells made competent according to the method of Spizizen (Anagnostopoulos, C. and Spizizen, J. (1961). J. Bacteriol. 81, 741-746) following plating on LB-agar plates with 20 μg/ml Kanamycin. Correct clones of final plasmid pUK57 were analyzed by restriction enzyme digest and sequencing.

Plasmid pUKA57: Type-II-Assembly Destination Bacillus Plasmid with alrA Gene

The alrA gene from B. subtilis with its native promoter region (SEQ ID 005) was PCR-amplified with oligonucleotides SEQ ID NO: 17 and SEQ ID NO: 18 comprising additional EcoRI sites. The backbone of pUK57S was PCR-amplified with oligonucleotides SEQ ID 015, SEQ ID 016 comprising additional EcoRI sites. After EcoRI and DpnI restriction, the two PCR fragments were ligated using T4 ligase (NEB) following transformation into B. subtilis Bs #056 cells made competent according to the method of Spizizen (Anagnostopoulos, C. and Spizizen, J. (1961). J. Bacteriol. 81, 741-746) following plating on LB-agar plates with 20 μg/ml Kanamycin and 160 μg/ml CDA (β-Chloro-D-alanine hydrochloride, Sigma Aldrich). Correct clones of final plasmid pUKA57 were analyzed by restriction enzyme digest and sequencing. The open reading frame of the alrA gene is opposite to the kanamycin resistance gene.

Plasmid pUA57: Type-II-Assembly Destination Bacillus Plasmid with alrA Gene

The alrA gene from B. subtilis with its native promoter region (SEQ ID NO: 5) was PCRamplified with oligonucleotides SEQ ID NO: 17 and SEQ ID NO: 18 comprising additional EcoRI sites. The backbone of pUK57S without the kanamycin resistance gene was PCR-amplified with oligonucleotides SEQ ID NO: 015 and SEQ ID NO: 19 comprising additional EcoRI sites. After EcoRI and DpnI restriction, the two PCR fragments were ligated using T4 ligase (NEB) following transformation into B. subtilis Bs #056 cells made competent according to the method of Spizizen (see above) following plating on LB-agar plates with 160 μg/ml CDA (13-Chloro-D-alanine hydrochloride, Sigma Aldrich). Correct clones of final plasmid pUA57 were analyzed by restriction enzyme digest and sequencing. The open reading frame of the alrA gene is opposite to the repU gene.

Protease expression plasmid pUKA58P The protease expression plasmid is composed of 3 parts—the plasmid backbone of pUKA57, the promoter of the aprEgene from Bacillus licheniformis from pCB56C (U.S. Pat. No. 5,352,604) and the protease gene of pCB56C (U.S. Pat. No. 5,352,604). The promoter fragment is PCR-amplified with oligonucleotides SEQ ID NO: 20 and SEQ ID NO: 21 comprising additional nucleotides for the restriction endonuclease BpiI. The protease gene is PCR-amplified from plasmid pCB56C (U.S. Pat. No. 5,352,604) with oligonucleotides SEQ ID NO: 22 and SEQ ID NO: 23 comprising additional nucleotides for the restriction endonuclease BpiI. The type-II-assembly with restriction endonuclease BpiI was performed as described (Radeck et al., 2017) and the reaction mixture subsequently transformed into B. subtilis Bs #056 cells made competent according to the method of Spizizen (see above) following plating on LB-agar plates with 20 μg/ml Kanamycin and 160 μg/ml CDA (I3-Chloro-D-alanine hydrochloride, Sigma Aldrich). Correct clones of final plasmid pUKA58P were analyzed by restriction enzyme digest and sequencing.

Bacillus Temperature Sensitive Deletion Plasmid

The plasmid pE194 is PCR-amplified with oligonucleotides SEQ ID 006 and SEQ ID 007 with flanking PvuII sites, digested with restriction endonuclease PvuII and ligated into plasmid pCE1 digested with restriction enzyme SmaI. pCE1 is a pUC18 derivative, where the BsaI site within the ampicillin resistance gene has been removed by a silent mutation. The ligation mixture was transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid is named pEC194S.

The type-II-assembly mRFP cassette is PCR-amplified from plasmid pBSd141R (accession number: KY995200)(Radeck et al., 2017) with oligonucleotides SEQ ID 008 and SEQ ID 009, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pDel003—aprE Gene Deletion Plasmid

The gene deletion plasmid for the aprEgene of Bacillus licheniformis was constructed with plasmid pEC194RS and the gene synthesis construct SEQ ID NO: 26 comprising the genomic regions 5′ and 3′ of the aprEgene flanked by BsaI sites compatible to pEC194RS. The type-II-assembly with restriction endonuclease BsaI was performed as described (Radeck et al., 2017) and the reaction mixture subsequently transformed into E. coli DH10B cells (Life technologies). Transformants were spread and incubated overnight at 37 C on LB-agar plates containing 100 μg/ml ampicillin. Plasmid DNA was isolated from individual clones and analyzed for correctness by restriction digest. The resulting aprE deletion plasmid is named pDel003.

pDel004—amyB Gene Deletion Plasmid

The gene deletion plasmid for the amyB gene of Bacillus licheniformis was constructed as described for pDel003, however, the gene synthesis construct SEQ ID 029 comprising the genomic regions 5′ and 3′ of the amyB gene flanked by BsaI sites compatible to pEC194RS was used. The resulting amyB deletion plasmid is named pDel004.

pDel005—sigF Gene Deletion Plasmid

The gene deletion plasmid for the sigF gene (spoIIAC gene) of Bacillus licheniformis was constructed as described for pDel003, however, the gene synthesis construct SEQ ID 032 comprising the genomic regions 5′ and 3′ of the sigF gene flanked by BsaI sites compatible to pEC194RS was used. The resulting sigF deletion plasmid is named pDel005.

pDel007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDel003, however, the gene synthesis construct SEQ ID 035 comprising the genomic regions 5′ and 3′ flanking the ywsC, ywtA (pgsC), ywtB (pgsA), ywtC (pgsE) genes flanked by BsaI sites compatible to pEC194RS was used. The resulting pga deletion plasmid is named pDel007.

pDel035—Alr Gene Deletion Plasmid

The gene deletion plasmid for the alr gene (SEQ ID 001) of Bacillus licheniformis was constructed as described for pDel003, however, the gene synthesis construct SEQ ID 038 comprising the genomic regions 5′ and 3′ of the alr gene flanked by BsaI sites compatible to pEC194RS was used. The resulting alr deletion plasmid is named pDel035.

pDel036—yncD Gene Deletion Plasmid

The gene deletion plasmid for the yncD gene (SEQ ID 024) of Bacillus licheniformis was constructed as described for pDel003, however, the gene synthesis construct SEQ ID NO: 41 comprising the genomic regions 5′ and 3′ of the yncD gene flanked by BsaI sites compatible to pEC194RS was used. The resulting yncD deletion plasmid is named pDel036.

### Example 1: Generation of B. licheniformis Enzyme Expression Strains

Bacillus licheniformis strains as listed in Table 1 were made competent as described above. For B. licheniformis strains with deletions in the alr gene or yncD, D-alanine was supplemented to all media and buffers. Protease expression plasmid pUKA58P was isolated from B. subtilis Bs #056 strain to carry the B. licheniformis specific DNA methylation pattern. Plasmids were transformed in the indicated strains and plated on LB-agar plates with 20 μg/μl kanamycin. Individual clones were analyzed for correctness of the plasmid DNA by restriction digest and functional enzyme expression was assessed by transfer of individual clones on LB-plates with 1% skim milk for clearing zone formation of protease producing strains. The resulting B. licheniformis expression strains are listed in Table 1.

TABLE 1
Overview on B. licheniformis expression strains

| B. licheniformis | | B. licheniformis |
|---|---|---|
| Expression strain | Expression plasmid | strain |
| BES#158 | pUKA58P | Bli#008 |
| BES#159 | pUKA58P | Bli#071 |
| BES#161 | pUKA58P | Bli#073 |

### Example 2: Cultivation of Bacillus licheniformis Protease Expression Strains

Bacillus licheniformis strains were cultivated in a fermentation process using a chemically defined fermentation medium.

The following macroelements were provided in the fermentation process:

| | | Concentration |
|---|---|---|
| Compound | Formula | [g/L initial volume] |
| Citric acid | C₆H₈O₇ | 3.0 |
| Calcium sulfate | CaSO₄ | 0.7 |
| Monopotassium phosphate | KH₂PO₄ | 25 |
| Magnesium sulfate | MgSO₄*7H₂O | 4.8 |
| Sodium hydroxide | NaOH | 4.0 |
| Ammonia | NH₃ | 1.3 |

The following trace elements were provided in the fermentation process:

| Trace element | Symbol | Concentration [mM] |
|---|---|---|
| Manganese | Mn | 24 |
| Zinc | Zn | 17 |
| Copper | Cu | 32 |
| Cobalt | Co | 1 |
| Nickel | Ni | 2 |
| Molybdenum | Mo | 0.2 |
| Iron | Fe | 38 |

The fermentation was started with a medium containing 8 g/I glucose. A solution containing 50% glucose was used as feed solution. The pH was adjusted during fermentation using ammonia. In both experiments, the total amount of added chemically defined carbon source was kept above 200 g per liter of initial medium. Fermentations were carried out under aerobic conditions for a duration of more than 70 hours.

At the end of the fermentation process, samples were withdrawn and the protease activity determined photometrically: proteolytic activity was determined by using Succinyl-Ala-Ala-ProPhe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage at 30° C., pH 8.6 TRIS buffer, resulting in release of yellow color of free pNA which was quantified by measuring at OD₄₀₅.

The protease yield was calculated by dividing the product titer by the amount of glucose added per final reactor volume. The protease yield of strain BES #158 was set to 100% and the protease yield of the other strains referenced to BES #158 accordingly. B. licheniformis expression strain BES #159, with the deletion of alr gene showed 9% improvement in the protease yield compared to B. licheniformis expression strain BES #158.

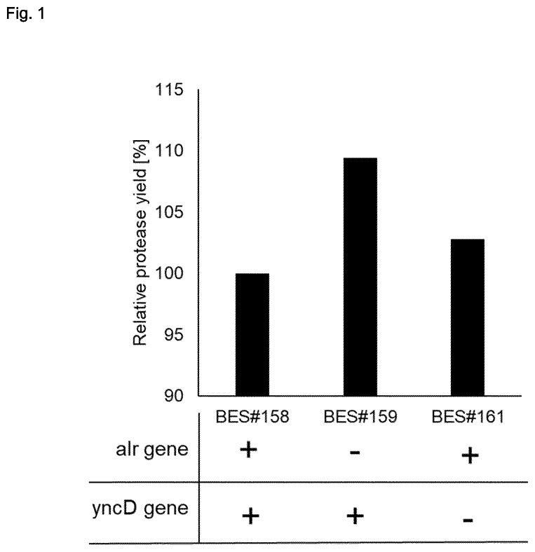

In contrast, the single knockout of the yncD gene showed a protease yield of 103% (see FIG. 1).

### Example 3: Alanine Racemase Activity of B. licheniformis Strains

Bacillus licheniformis cells were cultivated in LB media supplemented with 200 μg/ml D-alanine at 30° C. and harvested by centrifugation after 16 hours of cultivation by centrifugation. The cell pellet was washed twice using 1×PBS buffer and resuspended in 1×PBS supplemented with 10 mg/mL of lysozyme. Lysozyme treatment was performed for 30 min at 37° C. Complete cell lysis was performed using a ribolyser (Precellys 24). Cytosolic proteins were recovered by centrifugation and the supernatant was used for the determination of alanine racemase activity. The activity was determined using the method described by Wanatabe et al. 1999 (Watanabe et al., 1999; J Biochem.; 126(4):781-6). In brief, alanine racemase was assayed spectrophotometrically at 37° C. with D-alanine as the substrate. Conversion of D-alanine to L-alanine was determined by following the formation of NADH in a coupled reaction with L-alanine dehydrogenase. The assay mixture contained 100 mM CAPS buffer (pH 10.5), 0.15 units of L-alanine dehydrogenase, 30 mM D-alanine, and 2.5 mM NAD+, in a final volume of 0.2 ml. The reaction was started by the addition of alanine racemase after pre-incubation of the mixture at 37° C. for 15 min. The increase in the absorbance at 340 nm owing to the formation of NADH was monitored. One unit of the enzyme was defined as the amount of enzyme that catalyzed the racemization of 1 μmol of substrate per min. The activity was normalized using protein content measured by Bradford determination. Table 2 summarizes the alanine racemase activity of the different B. licheniformis strains.

TABLE 2
Alanine racemase activity in different B. licheniformis strains

| B. licheniformis | Genotype | Alr activity | Alr activity STD |
|---|---|---|---|
| strain | [alr genes] | [U/mg] | [U/mg] |
| Bli#008 | WT | 73.9 | 8.0 |
| Bli#071 | Dalr | <5 | n.a |
| Bli#073 | DyncD | 71.2 | 4.8 |

WT (wild-type): contains both endogenous chromosomal alanine racemase genes
Dalr: deletion of endogenous chromosomal alr gene
DyncD: deletion of endogenous chromosomal yncD gene
n.a: not available

Table 2 shows that Bacillus licheniformis strain Bli #071 with deleted alr gene shows complete loss of alanine racemase activity (<5 [U/mg], below background level). In contrast, Bacillus licheniformis strain Bli #073 with deleted yncD gene shows 71.2 U/mg of alanine racemase activity.

### Example 4: In Silica Assessment of the Presence of Alanine Racemase Genes in Bacterial Cells

An in silico analysis was carried out in order to identify all members of the alr gene family in bacterial cells using the EggNOG 5.0 database (Huerta-Cepas J, Szklarczyk D, Heller D, et al. eggNOG 5.0: a hierarchical, functionally and phylogenetically annotated orthology resource based on 5090 organisms and 2502 viruses. Nucleic Acids Res. 2019; 47(D1):D309-D314). A gene is considered to be a member of this family if, when searched against the collection of clusters of orthologous genes (COGs) provided by EggNOG 5.0, it has a significant alignment against COG0787. That is, COG0787 is the best hit, with an e-value>1e⁻¹⁰ and a score>100. This search can be done for multiple sequences using the eggNOG-mapper (Huerta-Cepas J, Forslund K, Coelho L P, et al. Fast Genome-Wide Functional Annotation through Orthology Assignment by eggNOG-Mapper. Mol Biol Evol. 2017; 34(8):2115-2122).

4214 different bacterial species were identified comprising between 1 to 5 alanine racemase genes. 829 species contain two different alanine racemase genes (not shown).

The identified alanine racemases were compared to the racemases from B. licheniformis. Table 3 provides an overview on YncD homologs with a high degree of identity to the B. licheniformis YncD polypeptide. Table 4 in the Examples section provides an overview on Alr homologs with a high degree of identity to the B. licheniformis Alr polypeptide.

TABLE 3
Overview on YncD homologs in different Bacillus species

| | | | Sequence identity to |
|---|---|---|---|
| | SEQ ID NO of the | | the B. licheniformis |
| | alanine racemase | Homolo- | YncD polypeptide |
| Species | polypeptide | gous to | SEQ ID NO 25 (%) |
| B. subtilis | 45 | YncD | 85.0 |
| B. pumilus | 54 | YncD | 77.1 |
| B. velezensis | 55 | YncD | 81.7 |
| B. atrophaeus | 56 | YncD | 85.5 |
| B. mojavensis | 57 | YncD | 76.0 |
| B. xiamenensis | 58 | YncD | 75.8 |
| B. zhangzhouensis | 59 | YncD | 75.1 |
| B. sonorensis | 60 | YncD | 85.5 |

TABLE 4
Overview on Alr homologs in different Bacillus species

| | | | Sequence | Sequence |
|---|---|---|---|---|
| | SEQ ID | | identity | identity to the |
| | NO | | to the | B. subtilis |
| | of the | | B. licheniformis | AlrA |
| | alanine | Homo- | Alr polypeptide | polypeptide |
| | racemase | logous | SEQ ID NO 2 | SEQ ID |
| Species | polypeptide | to | (%) | NO 4 (%) |
| B. subtilis | 4 | Alr | 68.3 | 100 |
| B. pumilus | 47 | Alr | 62.0 | 68.3 |
| B. velezensis | 48 | Alr | 66.0 | 62.8 |
| B. atrophaeus | 49 | Alr | 68.0 | 82.0 |
| B. mojavensis | 50 | Alr | 70.1 | 84.6 |
| B. xiamenensis | 51 | Alr | 62.0 | 92.5 |
| B. zhangzhouensis | 52 | Alr | 62.3 | 63.8 |
| B. sonorensis | 53 | Alr | 86.4 | 64.7 |

1. A method for producing at least one polypeptide of interest, said method comprising the steps of
a) providing a Bacillus host cell belonging to the species Bacillus licheniformis or Bacillus pumilus, in which the chromosomal alr gene has been inactivated and which comprises a plasmid comprising
1. at least one autonomous replication sequence,
2. a first polynucleotide encoding at least one polypeptide of interest, wherein said first polynucleotide is operably linked to a promoter, and
3. a second polynucleotide encoding an alanine racemase which is not native to the host cell, wherein said second polynucleotide is operably linked to a promoter, and
b) cultivating the host cell under conditions conducive for maintaining said plasmid in said host cell and conducive for expressing said at least one polypeptide of interest, thereby producing said at least one polypeptide of interest.

2. The method of claim 1, wherein step a) comprises the following steps:
a1) providing a Bacillus host cell belonging to the species Bacillus licheniformis or Bacillus pumilus,
a2) inactivating the chromosomal alr gene of said host cell, and
a3) introducing into said host cell a plasmid comprising
1. at least one autonomous replication sequence,
2. a first polynucleotide encoding at least one polypeptide of interest, wherein said first polynucleotide is operably linked to a promoter, and
3. a second polynucleotide encoding an alanine racemase which is not native to the host cell, wherein said second polynucleotide is operably linked to a promoter.

3. The method of claim 1 or 2, wherein the chromosomal alr gene has been inactivated by mutation.

4. The method of claim 3, wherein said mutation is a deletion of said chromosomal alr gen, or of fragment thereof.

5. The method of claim 1, wherein the Bacillus host cell belongs to the species Bacillus licheniformis.

6. The method of claim 5, wherein the Bacillus licheniformis host cell belongs to a Bacillus licheniformis species encoding a restriction modification system having a recognition sequence GCNGC.

7. The method of claim 1, wherein the alanine racemase which is not native to the host cell has at least 75% sequence identity to SEQ ID NO: 4.

8. The method of claim 6, wherein the alanine racemase which is not native to the host cell has at least 85%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99% at least 99.5%, or 100% sequence identity to SEQ ID NO: 4.

9. The method of claim 1, wherein the promoter which is operably linked to the polynucleotide encoding the alanine racemase is a constitutive promoter.

10. The method of claim 1, wherein the promoter which is operably linked to the polynucleotide encoding the alanine racemase is the promoter of the B. subtilis alrA gene, or a variant thereof having at least 80%, 85%, 90%, 93%, 95%, 98% or 99% sequence identity to said promoter.

11. The method of the claim 10, wherein the promoter of the B. subtilis alrA gene comprises a sequence as shown in SEQ ID NO: 46.

12. The method of claim 1, wherein the polypeptide of interest is an enzyme.

13. The method of claim 11, wherein the protease is an aminopeptidase (EC 3.4.11), a dipeptidase (EC 3.4.13), a dipeptidyl-peptidase or tripeptidyl-peptidase (EC 3.4.14), a peptidyl-dipeptidase (EC 3.4.15), a serine-type carboxypeptidase (EC 3.4.16), a metallocarboxypeptidase (EC 3.4.17), a cysteine-type carboxypeptidase (EC 3.4.18), an omega peptidase (EC 3.4.19), a serine endopeptidase (EC 3.4.21), a cysteine endopeptidase (EC 3.4.22), an aspartic endopeptidase (EC 3.4.23), a metalloendopeptidase (EC 3.4.24), or a threonine endopeptidase (EC 3.4.25).

14. The method of claim 1, wherein the at least one polypeptide of interest is secreted by the host cell into the fermentation broth.

15. The method of claim 1, further comprising step c) of purifying the polypeptide of interest.

16. A Bacillus host cell belonging to the species Bacillus licheniformis or Bacillus pumilus, in which the chromosomal air gene has been inactivated and which comprises a plasmid comprising
1. at least one autonomous replication sequence,
2. a first polynucleotide encoding at least one polypeptide of interest, wherein said first polynucleotide is operably linked to a promoter, and
3. a second polynucleotide encoding an alanine racemase which is not native to the host cell, wherein said second polynucleotide is operably linked to a promoter.

17. A fermentation broth comprising the Bacillus host cell of claim 16.
